(19) United States
(12) Patent Application Publication
(10) Pub. No.: US 2006/0094059 A1
(43) Pub. Date: May 4, 2006

(54) Methods for identifying new drug leads and new therapeutic uses for known drugs

(75) Inventors: John K. Westwick, San Ramon, CA (US); Marnie L. MacDonald, Pleasanton, CA (US); Helen Yu, Mountain View, CA (US); Stephen Owens, Martinez, CA (US); Stephen W. Michnick, Montreal (CA)

Correspondence Address: Isaac A. Angres, Suite 301, 2001 Jefferson Davis Highway, Arlington, VA, 22202, US

(73) Assignee: Odyssey Thera, Inc., San Ramon, CA (US)

(21) Appl. No.: 11/230,569

(22) Filed: Sep. 21, 2005

Related U.S. Application Data
(60) Provisional application No. 60/611,715, filed on Sep. 22, 2004.

(51) Int. Cl. C40B 40/10; G01N 33/53

(52) U.S. Cl. 435/7.1

(57) ABSTRACT

The screening system utilizes dynamic measurements of pathway activity to detect the activities of drugs within cellular pathways. The methods of the invention can be used to identify previously unknown drug activities and therapeutic uses, even for drugs that have been well characterized with standard biochemical assays. We demonstrated the utility of the invention by screening a portion of the known pharmacopeia. We identified dozens of drugs, previously or currently marked for a variety of indications, with surprising and previously-unsuspected activity against ‘hallmark’ cancer pathways. We also showed that over 20 of these drugs indeed have anti-proliferative activity in human tumor cells, underscoring the utility and predictability of the screening system. The methodology will extend the utility of the current pharmacopeia and provide the basis for de novo discovery of drugs with a broad range of therapeutic indications.

This application claims the priority benefit under 35 U.S.C. section 119 of U.S. Provisional Patent Application No. 60/611,715 entitled “Methods For Identifying New Drug Leads And New Therapeutic Uses For Known Drugs”, filed Sep. 22, 2004, which is in its entirety herein incorporated by reference.

### BACKGROUND OF THE INVENTION

Known drugs that are marketed for various therapeutic indications often have ‘hidden phenotypes’ resulting from unexpected or unintended activities on biochemical pathways in human cells. These observations of new and useful properties of known drugs are relatively infrequent and are usually discovered by serendipity.

A classical example is that of rapamycin (sirolimus), marketed as Rapamune®, which was approved for the treatment of immunosuppression in 1999. In the 1970's, rapamycin was found by the NCI to have potential anticancer properties. Now, over 30 years later, an analog of rapamycin known as CCI-779 is finally in clinical trials for the treatment of breast and renal carcinoma. As recently as 2000, rapamycin was also found to prevent restenosis when used in coated (rapamycin-eluting) stents; the stents are sold by Cordis Corp. for that purpose. These properties result from the underlying activity of rapamycin on biochemical pathways: namely, its ability to activate a pathway leading to apoptosis. Another example is that of thalidomide, the drug originally developed as an anti-emetic. Thalidomide, which is in clinical development for the treatment of multiple myeloma, is likely to have a broad spectrum of anti-cancer activity as a result of its ability to block a pathway that is a hallmark of the cancer cell.

If there were a reliable and systematic method to identify such useful biological properties, such observations could be made on a large scale. This would have important benefits not only for patients in need of new remedies, but also for the pharmaceutical industry.

Perturbations in signal transduction pathways are known to underlie the mechanisms of action of most if not all drugs. In principle, unexpected biologic activities of drugs could be found by testing their activities directly against the pathways that control the behavior of the living cell. If an unexpected activity is found against a pathway that is known to be linked to a disease, the drug can be tested in phenotypic assays, model organisms and other model systems to determine if it has an effect on that disease. Since known drugs have established safety profiles and pharmacodynamic properties, if the drug shows promise of being effective in the new disease indication it can be rapidly advanced into clinical trials for the treatment of patients with that disease. Therefore we sought to develop a rapid, pathway-based system in living cells for rapidly identifying unexpected activities of drugs on a large scale. The advantages of using a cell-based system are that drugs can be studied in the context of the complex biology of the whole cell.

To date, cell-based screening approaches have relied either on phenotypic screens, reporter gene assays, or mRNA profiling. For a summary of such approaches, see the Disease Proteomics reference. For example, cells can be treated with individual drugs or elements of chemical libraries and a phenotype can be measured, such as growth, apoptosis, migration, cell cycle arrest, etc. Phenotypic screens have been widely used in recent years but do not provide an indication of the underlying mechanism by which drugs cause the phenotypic change.

Reporter gene assays have also been used to identify the activities of compounds and drugs against biochemical pathways in living cells. Reporter gene assays couple the biological activity of a target to the expression of a readily detected enzyme or protein reporter, allowing monitoring of the cellular events associated with signal transduction and gene expression. Based upon the fusion of transcriptional control elements to a variety of reporter genes, these systems “report” the effects of a cascade of signaling events on gene expression inside cells. Synthetic repeats of a particular response element can be inserted upstream of the reporter gene to regulate its expression in response to signaling molecules generated by activation of a specific pathway in a live cell. Such assays have proven useful in primary and secondary screening of chemical libraries and drug leads. However, such assays only measure the consequence of pathway activation or inhibition and not the site of action of the compound.

Microarrays allow measurements of gene expression patterns on a large scale. Following a drug treatment, messenger RNA is isolated from a cell or tissue; and the expression patterns of the mRNA in the absence and presence of the drug are compared. Identifying groups of genes that are stimulated or repressed in response to specific conditions or treatments is a useful way to begin to unravel the cellular mechanisms of drug response. However, changes in the level of particular mRNA molecules do not always correlate directly with the level or activity of any corresponding protein at a single point in time. Furthermore, many proteins undergo post-translational modifications and protein-protein interactions, which may affect the functions and activities of proteins within a tissue or cell. Consequently, gene chip experiments are not always predictive of biological activity.

In sum, such approaches do not enable an understanding of the mechanisms of action of drugs at their site of activity within biochemical pathways. It would be preferable to directly probe the networks of living human cells. Direct measures of specific events within signaling pathways would eliminate the problems associated with interpretation of transcriptional profiles. Unlike transcriptional reporter assays, the information obtained by monitoring a protein modification or its interactions reflects the effect of a drug on a particular branch or node of a cell signaling pathway, not its endpoint.

In making the present invention, our central premise was that (a) the biological and biochemical effects of drugs can be studied with living cells; (b) one can determine the unexpected effects of drugs on cellular pathways by probing those pathways directly, at the level of specific proteins, following drug treatment; (c) the effects of drugs on their targets will propagate through or between functional modules, inducing spatial and temporal changes in proteins downstream of the target of a drug (FIG. 1); (d) such changes can be quantified by measuring changes in protein-protein complexes or interactions ‘downstream’ of the site(s) of action of the drug; and (e) such changes will be dynamic, that is, they will occur transiently within minutes—or at most, within hours—after drug treatment of the cells.

Changes in protein-protein complexes (interactions) could be effected by a variety of biochemical events within the module (e.g. post-translational modification, allosteric transition, protein degradation or de novo protein synthesis, protein stabilization or destabilization, or protein translocation), wherein the change propagates through or between modules from the drug target, resulting in a perturbation of a protein-protein complex. In the context of the invention, we use the terms “protein-protein interaction” and “protein-protein complex” interchangeably. An interaction between proteins is reflected in the presence of a complex between the proteins, and the amount and/or location of the complex is altered by biochemical events that stimulate or inhibit the pathways that influence the proteins in question. We hypothesized that temporal, drug-induced changes in the amount, subcellular location, or post-translational modification status of proteins within a dynamic complex within a pathway could be detected by directly measuring particular complexes, as protein-protein pairs, in human cells following drug treatment.

We systematically applied this strategy to the known pharmacopeia in order to identify drugs that are capable of modulating the activity of the oncogenic pathways underlying the cancer phenotype. By ‘known drug’ and ‘known pharmacopeia’ we mean drugs currently or previously administered to patients. We screened a portion of the known pharmacopeia and identified dozens of drugs, previously or currently marked for a variety of indications, with surprising and previously-unsuspected activity against ‘hallmark’ cancer pathways. We then showed that over 20 of these drugs indeed have anti-proliferative activity in tumor cells, underscoring the utility and predictability of the screening approach. The drugs we identified represent potential new treatments for cancer in man. Importantly, the strategy and methods presented herein represent an entirely novel system for therapeutic discovery on a large scale.

### SUMMARY OF THE INVENTION

We have invented a powerful new method for identifying compounds with new and useful biological activities. The methods of the invention can be used to identify previously unknown drug activities, even for drugs that have been well characterized with standard biochemical assays.

The screening system utilizes dynamic measurements of pathway activity to detect the activities of drugs within cellular pathways. The invention has commercial importance for the pharmaceutical industry. If drugs that are known to be safe can be found to have new indications, they can be rapidly advanced to clinical trials to demonstrate efficacy for the new indications. Also, if drugs that are known to be safe have failed to demonstrate efficacy for their originally intended indication, they may still be rescued for use in a new therapeutic indication. Finally, drugs that have adverse effects when used at specific doses or in chronic administration may still be tolerable if used in a new indication or a new dosing regimen.

The methodology will extend the utility of the current pharmacopeia and provide the basis for de novo discovery of drugs with a broad range of therapeutic indications. In the present invention we identified antiproliferative activities for over 20 known drugs including drugs previously used for the treatment of congestive heart failure, hypertension, hypercholesterolemia, asthma, infection (antibiotic, antiprotozoan, antihelminthic, antifungal), emesis, migraine, psychosis, dementia, and other common conditions. The drugs we identified as having activity on cancer pathways include sertraline (Zoloft), terfenadine (Seldane), atorvastatin (Lipitor), fenofibrate (Tricor) and other well-known drugs currently or previously marketed for a wide range of non-cancer indications. Some of these activities have been previously suspected whereas others are completely unsuspected.

These activities are linked to the ability of these drugs to inhibit one or more of the key pathways contributing to the cancer phenotype.

Our success in identifying drugs with potential anti-cancer activities is likely due to the uniquely informative whole-cell assay approaches we employed here; in particular, the ability to assess multiple pathway activities of drugs in human cells that have the requisite intracellular machinery. The approach can be applied to a wide range of chronic and acute diseases in man by simply probing other pathways linked to those diseases.

### OBJECTS AND ADVANTAGES OF THE INVENTION

It is an object of the present invention to provide methods for the identification of new therapeutic uses for existing drugs.

A further object of this invention is to provide methods, assays and compositions useful for drug discovery on a large scale.

The present invention has the advantage of being broadly applicable to any disease or medical condition, drug target class or agent.

The present invention has the advantage of being independent of the primary or intended or original target of a drug or drug candidate.

The present invention has the advantage of being applicable to any therapeutic indication.

The present invention has the advantage of being applicable to any cell type or disease model system or organism on a genome-wide scale.

The present invention has the advantage of being performed in high throughput and can be completely automated.

### BRIEF DESCRIPTION OF THE DRAWINGS

FIG. 1 Schematic of the relationship between a drug target and a cellular assay in the present invention. Effects of drugs on cellular pathways can be determined by measuring protein interactions and/or modifications ‘downstream’ of a drug target. Shown in red are network ‘nodes’; wherein each node is a protein-protein complex.

FIG. 2 Pathways probed in the present invention. Protein-protein complexes, comprised of the signaling proteins that are outlined in red, formed the basis for the construction of assays in living cells. Physical interactions between proteins are indicated by arrows.

FIG. 3A Examples of drug effects on BCL-xL:BIK and PIN1:JUN complexes in human cells. Photomicrographs show the effects of fenofibrate on BclxL:BIK complexes and the effect of niclosamide on PIN1:JUN complexes, as assessed with protein-fragment complementation assays. The drugs cause a decrease in the level of the protein-protein complexes as assessed by a decrease in the intensity of the fluorescence in the assays.

FIG. 3B Examples of drug effects on p27:Ubiquitin and CyclinD1:CDK4 complexes in human cells. Photomicrographs show the effects of fenofibrate on p27:Ubiquitin complexes and on CyclinD1:CDK4 complexes, as assessed with protein-fragment complementation assays. Fenofibrate caused a decrease in the level of the protein-protein complexes Fenofibrate caused a decrease in the level of these protein-protein complexes as assessed by a decrease in the intensity of the fluorescence in the assays.

FIG. 3C Examples of drug effects on AKT1:p27 and Cofilin:LIMK2 in human cells. Photomicrographs show the effects of fenofibrate on the protein-protein complexes, as assessed with protein-fragment complementation assays. Fenofibrate caused a decrease in the level of these protein-protein complexes as assessed by a decrease in the intensity of the fluorescence in the assays.

FIG. 3D. Examples of drug effects on HSP90:CDC37 and HSP90:Eef2k in human cells. Photomicrographs show the effects of niclosamide on the protein-protein complexes as assessed with protein-fragment complementation assays. Niclosamide caused a decrease in the level of these protein-protein complexes as assessed by a decrease in the intensity of the fluorescence in the assays.

FIG. 3E Examples of drug effects on Ras:Raf in human cells. Photomicrographs show the effects of promazine, sanguinarine, desispramine, metergoline, and tamoxifen citrate on Ras:Raf complexes, as assessed with protein-fragment complementation assays. The drugs caused a decrease in the level of the Ras:Raf complexes, as assessed by a decrease in the intensity of the fluorescence compared with the vehicle alone; the drugs also caused a change in the subcellular location of the complexes, as assessed by an obvious change in the subcellular pattern of the fluorescence. In the control cells (vehicle only) the Ras:Raf complexes were localized at the cell membrane; the drugs caused a redistribution of the complexes to an intracellular structure.

FIG. 3F Examples of drug effects on CDC42:PAK4 in human cells. Photomicrographs show the effects of terfenadine, bepridil, and metergoline, on CDC42:PAK4 complexes, as assessed with protein-fragment complementation assays. The drugs caused a decrease in the level of these protein-protein complexes as compared with the vehicle alone, as assessed by a decrease in the intensity of the fluorescence in the assays.

FIG. 3G. Examples of drug effects on the post-translational modification status of ERK (MAPK) in the presence of VEGF. Photomicrographs show the effects of fenofibrate and niclosamide on phospho-ERK, as assessed with immunofluorescence assays using phospho-ERK-specific antibodies. The drugs caused a decrease in the level of phospho-ERK as compared with the vehicle alone, as assessed by a decrease in the intensity of the fluorescence in the assay.

FIG. 4 Antiproliferative activity of fenofibrate vs. an analog. The ability of fenofibrate to reduce the proliferation of PC-3 cells is shown. An analog, WY-14643, had no effect on proliferation as assessed by a MTT assay, demonstrating a structure-activity relationship in these cellular assays. Dose dependence for fenofibrate (triplicate assays) is shown in the proliferation assay as compared to the DMSO control. Also shown are images of the MTT assay wells, and phase contrast images of the treated cells vs. the untreated (DMSO) control showing that fenofibrate reduced the cell count as compared with the control.

FIG. 5 Antiproliferative activity of terfenadine (Seldane). Dose dependence for terfenadine (triplicate assays) is shown in the MTT proliferation assay as compared to the untreated (DMSO) control. Also shown are images of the MTT assay wells and phase contrast images of the treated cells vs. the untreated (DMSO) control, showing that terfenadine reduced the cell count as compared with the control.

FIG. 6 Antiproliferative activity of sertraline (Zoloft). Dose dependence for sertraline (triplicate assays) is shown in the MTT assay as compared to the DMSO control. Also shown are images of the MTT assay wells and phase contrast images of the treated cells vs. the untreated (DMSO) control, showing that sertraline reduced the cell count as compared with the control.

FIG. 7 Antiproliferative activity of cinnarazine. Dose dependence for cinnarizine (triplicate assays) is shown in the MTT assay as compared to the DMSO control. Also shown are images of the MTT assay wells and phase contrast images of the treated cells vs. the untreated (DMSO) control, showing that cinnarazine decreased the cell count as compared with the control.

FIG. 8 Antiproliferative activity of isoreserpine. Dose dependence for isoreserpine (triplicate assays) is shown in the MTT assay as compared to the DMSO control. Also shown are images of the MTT assay wells and phase contrast images of the treated cells vs. the DMSO control, showing that isoreserpine decreased the cell count as compared with the control.

FIG. 9 Antiproliferative activity of clotrimazole. Dose dependence for clotrimazole (triplicate assays) is shown in the MTT assay as compared to the DMSO control. Also shown are images of the MTT assay wells and phase contrast images of the treated cells vs. the DMSO control, showing that clotrimazole decreased the cell count as compared with the control.

FIG. 10 Antiproliferative activity of atorvastatin (Lipitor). Dose dependence for atorvastatin (triplicate assays) is shown in the MTT assay as compared to the DMSO control. Also shown are images of the MTT assay wells and phase contrast images of the treated cells vs. the DMSO control, showing that atorvastatin decreased the cell count as compared with the control.

FIG. 11 Positive predictive value of the process. 960 individual drugs were tested in 4 different assays in human cells according to the present invention. Drugs that showed activity in any one of the four assays were then tested at a single concentration to determine if they were capable of reducing the proliferation of PC-3 cells, as assessed by a reduction in cell number to less than 80% of or less than 50% of the control (untreated) cells. The number of assay hits that proved to have antiproliferative activity is shown.

### DETAILED DESCRIPTION OF THE INVENTION

Perturbations in signal transduction pathways are known to underlie the mechanisms of action of most if not all drugs. Drugs that are capable of re-routing the abnormal circuits of the disease cell should in principle be capable of restoring the phenotype of the normal or healthy cell. The phenotype of a cell is, in turn, controlled at a high level by biochemical pathways that regulate the expression and activity of proteins. Hence, the key to identifying drugs that are capable of re-routing cellular circuits is the ability to detect specific activities of drugs within, and on, the pathways of living cells. Our objective was to establish a systematic, high-throughput process for the identification of agents capable of rerouting the circuits of living cells.

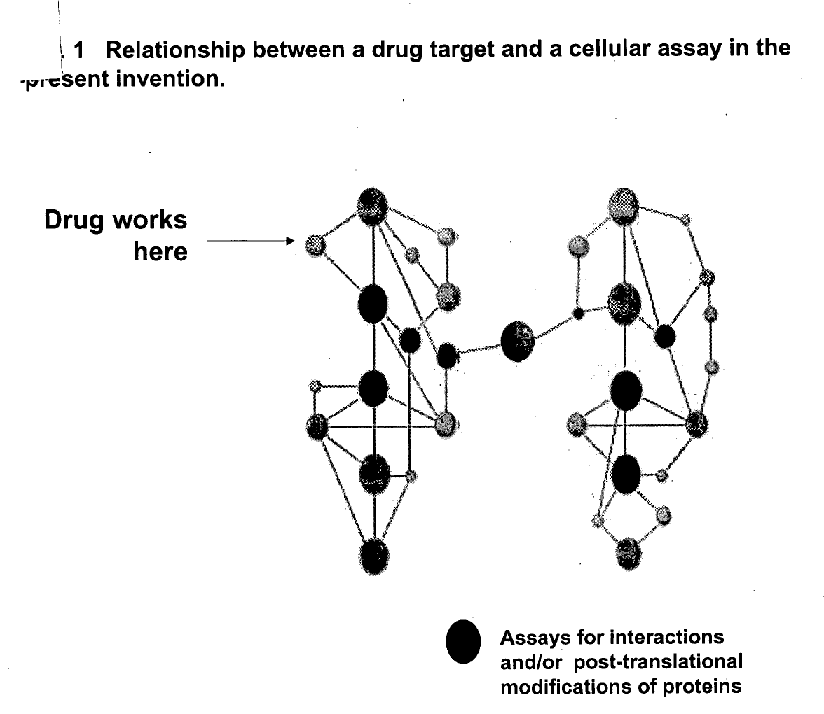

The invention is based on the concept of a cellular network as a series of interconnected pathways involving physical connections between proteins, as depicted in FIG. 1. The rationale underlying the invention is as follows. A pathway is a series of steps, with each step occurring at a particular point in time (with the first step preceding the second step which precedes the third step, etc.) and in space (for example, starting with a receptor at the cellular membrane and proceeding to a transcription factor in the cell nucleus). In our model, each step involves the association or dissociation of proteins. If the pathway is a signal transduction pathway, activation of the pathway—for example, by binding of an agonist to a receptor—initiates a cascade of events by which an external signal is transduced to the nucleus. These events involve the interactions of proteins which modify their activities. These changes are dynamic, beginning within minutes of drug treatment. The ultimate consequence of these biochemical events is a change in cell behavior: growth, division, apoptosis, migration, differentiation, metastasis, or another behavior that is characteristic of the cell under study. Drugs that block a particular step would lead to inhibition of the steps ‘downstream’ of the original site of action of the drug. Conversely, drugs that activate a particular step would lead to activation of the steps ‘downstream’ of the original site of action of the drug. It follows that dynamic measurements of the proteins within selected functional pathways should enable the identification of drugs that affect, in a desired way, the activities of those pathways.

Typically, assessing the activity of individual proteins involves in vitro measurements of enzyme activity (kinases, phosphatases, proteases, hydrolases, etc.) and/or ligand binding, in the case of receptors. We sought instead to measure the physical/chemical changes in proteins that precede or coincide with such activity changes. Such physical/chemical changes involve interactions between proteins that lead to the formation and movement of protein-protein complexes; and the post-translational modifications that result from those interactions and movements. Moreover, we sought to make such measurements in intact cells before and after treating the cells with a drug.

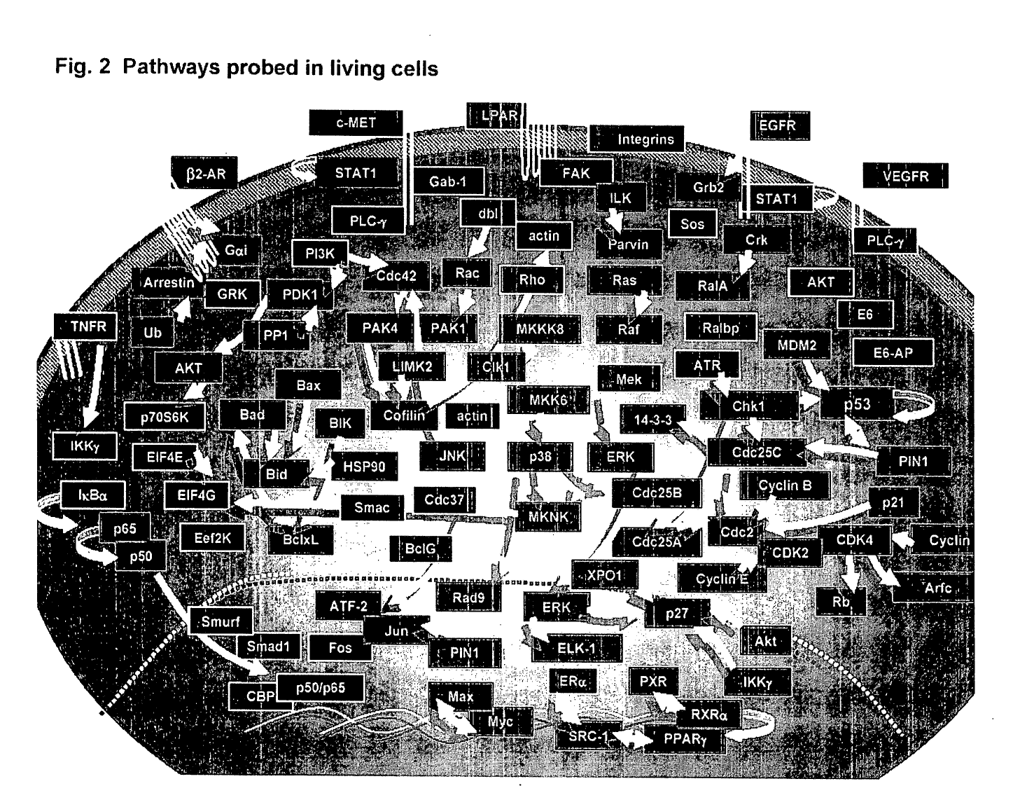

Cancer cells have defects in regulatory circuits that govern the behavior of healthy cells. For cancer, these defects occur in the regulatory circuits that govern normal cell proliferation and homeostasis. Hanahan and Weinstein have outlined the essential alterations in cell physiology that collectively dictate malignant growth. These include: self-sufficiency in growth signals; insensitivity to growth-inhibitory (antigrowth) signals; evasion of programmed cell death (apoptosis); limitless replicative potential; sustained angiogenesis; and tissue invasion and metastasis. Each of these physiologic changes is a result of alterations in the behavior of the proteins that control the underlying biochemical pathways. Such alterations are due to mutational changes as well as changes in the expression level of various proteins. As a result, the pathways controlling mitogenesis, apoptosis, the cell cycle, invasion and metastasis are abnormally regulated. It follows that a successful therapeutic strategy involves re-routing the abnormal circuits of the cancer cell such that normal cellular behavior is restored. Therefore we sought to construct live cell-based assays for key cancer pathways (FIG. 2) and to use these assays to screen for drugs capable of regulating these pathways. We started with the known pharmacopeia in order to identify potential hidden phenotypes of known drugs. The strategy and the methods provided herein will also be useful for de novo drug discovery.

Experimental Design

The strategy involved constructing dynamic, pathway-specific screening assays in live cells; treating the cells with a drug; and assaying for previously-unsuspected activities of drugs on the pathway(s) of interest. We constructed assays in human cells to ‘read out’ the activity of pathways known to be involved in the pathways of interest. The pathways were selected to represent the key hallmarks of the cancer cell outlined above.

These include such well characterized pathways as the MAP kinase pathway (phosphoprotein ERK); the ras/raf oncogenic pathway (ras oncogene and raf kinase); cell cycle pathways (Cyclin DI, cyclin-dependent kinase CDK4, cell cycle progression kinase CDC2, transcription factor c-MYC and cell cycle regulatory protein p27); apoptotic pathways (BID, BAD and BCL-xL); the proteasome and chaperone systems (Ubiquitination; heat shock protein HSP90); and the actin cytoskeleton (cofilin, the CDC42 effector kinase PAK4, and the LIM kinase LIMK2).

The proteins selected for assay construction are shown in Table 1 with a description of their function. Although the biochemical functions of these proteins have been well characterized, the prior art is silent on the use of live cell assays for these proteins in the context of drug discovery.

To achieve our objectives, it was necessary to satisfy the following criteria: (1) Assays must be constructed that are sufficiently sensitive to detect the dynamic or transient changes in individual proteins, or protein-protein complexes, that occur upon pathway activation or inhibition; (2) The chosen assays must be capable of reading out the activity of a pathway in an intact cell, but the assay should not disrupt the biology of the cell or the pathway of interest; (3) Activation or inhibition of a particular pathway must be readily detectable and quantifiable; (4) Ideally, the methods will be suitable for scale-up and automation.

The effects of drugs on cellular pathways of interest were determined by measuring changes in the level and/or the subcellular location and/or post-translational modification status of protein complexes in cells following drug treatment. The assays we used can be performed with automated instrumentation, using automated microscopy, automated image analysis, or alternative fluorescence instrumentation which is described in detail below.

We used these methods to mine the known pharmacopeia for drugs with previously-unsuspected activity against cancer-related pathways. Specifically, we screened drugs that have been used to treat patients for diseases other than cancer and screened for novel activities on cancer pathways. Hits from our cell-based screens were tested for antiproliferative activity in up to 5 different human tumor cell lines, as follows. First, drugs with significant activity in the cellular screen(s) were tested for their ability to block proliferation of a human tumor cell line (PC-3) at an initial concentration of 10 micromolar. For drugs with antiproliferative activity in PC-3 cells, dose-response curves to determine the IC50 for antiproliferative activity and additional tumor cell lines were tested to determine the breadth of activity. Methods for assessing the antiproliferative activity of the cell-based drug ‘hits’ are described below.

Based on our initial screens of 960 known drugs and natural products, we identified over 20 drugs with unexpected activity on cancer-related pathways. For the majority of these, the observed activity on cancer pathways represents a completely novel finding. These drugs are being further developed for potential clinical use in the treatment of cancer and other neoplasms.

Methods for the Construction of Cell-Based Assays

To construct dynamic assays for protein-protein complexes, we used a protein-fragment complementation assay strategy (PCAs) in intact human cells. (A variety of other suitable assays, and reporter options, are described in detail below.) The principle of PCA is that complementary polypeptide fragments (F[1] and F[2]) of a reporter protein or enzyme will fold into an active form only if fused to two proteins which interact and bring the complementary fragments of the reporter protein into proximity. Reconstitution of the fragments of the reporter protein generates a fluorescent signal that can be quantified in intact cells, and the amount of the signal is proportional to the amount of the protein-protein complex used in constructing the assay. Given a suitable reporter type, the subcellular location of the complex can also be measured.

We studied protein-protein interactions within key pathways regulating cell homeostasis. Specifically, we studied interacting proteins within pathways representing the processes of cell cycle control, DNA damage response, apoptosis, molecular chaperones, cytoskeletal regulation, proteasomal degradation, mitogenesis, inflammation, and nuclear hormone receptor activation. Proteins used to construct these particular assays are summarized in Tables 1 and 2. For reporting out protein-protein interactions, we constructed PCAs based on fragments of fluorescent proteins; however, many other reporter proteins are suitable for use with PCA (see U.S. Pat. No. 6,270,964 and the References herein) including enzymatic reporters such as dihydrofolate reductase (DHFR), beta-lactamase, luciferase, beta-galactosidase and others which are discussed in more detail below.

We also studied post-translational modifications of particular proteins within the same pathways in response to drugs. An ever-increasing assortment of phospho-specific antibodies enables probing of the phosphorylation status of individual proteins in tissues or cells, either in whole tissues or cells or in vitro in cell or tissue extracts. Phospho-specific antibodies are distributed by life sciences product companies including BD Biosciences (www.bdbiosciences.com), Cell Signaling Technology (www.cellsignal.com) and a variety of other companies. These antibodies are directed specifically against phosphorylated antigens and do not recognize the unphosphorylated form of the protein. Methods suitable for such assays are well known to those skilled in the art of cell biology. Immunofluorescence methods—combined with robotic systems and automated fluorescence microscopy—offer the additional potential for the development of high-throughput screens that combine the biological advantages of intact cell detection with scalable, automated, high-throughput methods.

Although the individual techniques we employed have been widely used in mechanistic biochemical research, the prior art is silent on the use of such methods to identify hidden phenotypes of drugs. We sought to apply such methods to identify new indications for known drugs and to carry out de novo drug discovery on a broad scale.

Protein-Fragment Complementation Assays (PCA)

Reporter fragments for PCA were generated by oligonucleotide synthesis (Blue Heron Biotechnology, Bothell, Wash.), starting with the sequence of yellow fluorescent protein (YFP). First, oligonucleotides coding for polypeptide fragments YFP[1]and YFP[2] (corresponding to amino acids 1-158 and 159-239 of YFP) were synthesized. Next, PCR mutagenesis was used to generate the mutant fragments IFP[1] and IFP[2]. The IFP[1] fragment corresponds to YFP[1]-(F46L, F64L, M153T) and the IFP[2] fragment corresponds to YFP[2]-(V163A, S175G). These mutations have been shown to increase the fluorescence intensity of the intact YFP protein (Nagai et al., 2002). The YFP[1], YFP[2], IFP[1] and IFP[2] fragments were amplified by PCR to incorporate restriction sites and a linker sequence, described below, in configurations that would allow fusion of a gene of interest to either the 5′- or 3′-end of each reporter fragment. The reporter-linker fragment cassettes were subcloned into a mammalian expression vector (pcDNA3.1Z, Invitrogen) that had been modified to incorporate the replication origin (oriP) of the Epstein Barr virus (EBV). The oriP allows episomal replication of these modified vectors in cell lines expressing the EBNA1 gene, such as HEK293E cells (293-EBNA, Invitrogen). Additionally, these vectors still retain the SV40 origin, allowing for episomal expression in cell lines expressing the SV40 large T antigen (e.g. HEK293T, Jurkat or COS). The integrity of the mutated reporter fragments and the new replication origin were confirmed by sequencing.

PCA fusion constructs were prepared for a proteins known to participate in cellular pathways that have been described in the scientific literature as being linked to cancer. The selection of protein-protein complexes used, and the rationale for their use, is provided in Table 1 and the gene identifiers for the cDNAs used in assay construction are provided in Table 2. The full coding sequence for each gene of interest was amplified by PCR from a sequence-verified full-length cDNA. Resulting PCR products were column purified (Centricon), digested with appropriate restriction enzymes to allow directional cloning, and fused in-frame to either the 5′ or 3′-end of YFP[1], YFP[2], IFP[1] or IFP[2] through a linker encoding a flexible 10 amino acid peptide (Gly.Gly.Gly.Gly.Ser)2. The flexible linker ensures that the orientation or arrangement of the fusions is optimal to bring the reporter fragments into close proximity (Pelletier et al., 1998). Recombinants in the host strains DH5-alpha (Invitrogen, Carlsbad, Calif.) or XL1 Blue MR (Stratagene, La Jolla, Calif.) were screened by colony PCR, and clones containing inserts of the correct size were subjected to end sequencing to confirm the presence of the gene of interest and in-frame fusion to the appropriate reporter fragment. A subset of fusion constructs were selected for full-insert sequencing by primer walking. DNAs were isolated using Qiagen MaxiPrep kits (Qiagen, Chatsworth, Calif.). PCR was used to assess the integrity of each fusion construct, by combining the appropriate gene-specific primer with a reporter-specific primer to confirm that the correct gene-fusion was present and of the correct size with no internal deletions.

Transfections and Cell Preparation

HEK293 cells were maintained in MEM alpha medium (Invitrogen) supplemented with 10% FBS (Gemini Bio-Products), 1% penicillin, and 1% streptomycin, and grown in a 37° C. incubator equilibrated to 5% CO₂. Approximately 24 hours prior to transfections cells were seeded into 96 well ploy-D-Lysine coated plates (Greiner) using a Multidrop 384 peristaltic pump system (Thermo Electron Corp., Waltham, Mass.) at a density of 7,500 cells per well. Up to 100 ng of the complementary YFP or IFP-fragment fusion vectors were co-transfected using Fugene 6 (Roche) according to the manufacturer's protocol. A list of the selected protein-protein complexes (PCA pairs) screened in this study, is in Table 2. Following 24 or 48 hours of expression, cells were screened against the selected drugs as described below.

For several PCAs, stable cell lines were generated. HEK293 cells were transfected with a first fusion vector and stable cell lines were selected using 100 μg/ml Hygromycin B (Invitrogen). Selected cell lines were subsequently transfected with the second, complementary fusion vector, and stable cell lines co-expressing the complementary fusions were isolated following double antibiotic selection with 50 μg/ml Hygromycin B and 500 μg/ml Zeocin. For all cell lines, the fluorescence signals were stable over at least 25 passages (data not shown). Approximately 24 hours prior to drug treatments, cells were seeded into 96 well ploy-D-Lysine coated plates (Greiner) using a Multidrop 384 peristaltic pump system (Thermo Electron Corp., Waltham, Mass.).

Assessing Drug Activity on Protein-Protein Complexes

Drugs were screened in duplicate wells at a concentration of 10 micromolar. All liquid handling steps were performed using the Biomek FX platform (Beckman Instruments, Fullerton, Calif.). Cells expressing the PCA pairs were incubated in cell culture medium containing drugs for 90 min. and 8 hours, or in the case of pre-stimulation with camptothecin (CPT) for 16-18 hours. For some assays cells were treated with known pathway agonists immediately prior to the termination of the assay. Following drug treatments cells were stained with 33 micrograms/ml Hoechst 33342 (Molecular Probes) and fixed with 2% formaldehyde (Ted Pella) for 10 minutes. In some cases cells were simultaneously stained with Hoechst and with 15 micrograms/ml TexasRed-conjugated Wheat Germ Agglutinin (WGA; Molecular Probes), and then fixed. Cells were subsequently rinsed with HBSS (Invitrogen) and maintained in the same buffer during image acquisition.

YFP, Hoechst, and Texas Red fluorescence signals were acquired using the Discovery-1 automated fluorescence imager (Molecular Devices, Inc.) equipped with a robotic arm (CRS Catalyst Express; Thermo Electron Corp., Waltham, Mass.). The following filter sets were used to obtain images of 4 non-overlapping populations of cells per well: excitation filter 480/40 nm, emission filter 535/50 nm (YFP); excitation filter 360/40 nm, emission filter 465/30 nm (Hoechst); excitation filter 560/50 nm, emission filter 650/40 nm (Texas Red). All treatment conditions were run in duplicate yielding a total of 8 images for each wavelength and treatment condition.

TABLE 1
Assays used to demonstrate the invention and their rationale

| Assay | Brief Assay Description |
|---|---|
| BclxL: Bad | Key node for apoptotic signaling. Bad complexes with BclxL and Bcl-2 block the anti-apoptotic activity of |
| | the latter two proteins |
| BAD: BID | Indicates apoptotic activity |
| BIK: BCL-xL | Key node for apoptotic signaling. Bid complexes with BclxL and Bcl-2 block the anti-apoptotic activity of |
| | the latter two proteins |
| Hsp90: CDC37 | HSP90 is key chaperone regulating protein stability/activity/half-life. CDC37 is co-chaperone; determines |
| | activity and client protein selectivity |
| CDC42: PAK4 | small GTPase/kinase signaling node. PAK4 is CDC42 effector; transmits the signal from the molecular |
| | switch to downstream substrates such as LIMK, BAD |
| CyclinD: Cdk4 | key cell cycle control node |
| Chk1: CDC25C +CPT | Chk kinases regulate CDC25 phosphatases; activation indicates cell cycle checkpoint activation; CPT |
| | (camptothecin) topoisomerase inhibitor causes DNA damage and activates checkpoints |
| Chk1: CDC25A +CPT | Chk kinases regulate CDC25 phosphatases; activation indicates cell cycle checkpoint activation |
| Chk1: CDC25C | Chk kinases regulate CDC25 phosphatases; activation indicates cell cycle checkpoint activation |
| Cofillin: LIMK2 | LIM kinases phospohorylate cofilin and regulate cytoskeletal dynamics |
| Hsp90: Eef2k | translation factor-controlling kinase Eef2k is HSP client protein |
| EGFR: Grb2 | receptor tyrosine kinase: adaptor protein complex; indicates activated receptor |
| Erk2: Elk1 | ERK mitogen-activated protein kinase interacts with and phosphorylates the Elk-1 (Ets family) |
| | transcription factor |
| H-Ras: Raf | small GTPase/kinase signaling node. Ras is commonly mutated human oncogene; activates ERK/MAP |
| | kinase path among others; downstream from receptor tyrosine kinases and some G-proteins |
| MAX: MYC | c-Myc is a transcription factor and human proto-oncogene. Activity correlates with cell cycle progression |
| PAK4: Cofilin | complex of upstream activator PAK4 with downstream effector cofiin; regulates actin cytoskeleton |
| Smad3: HDAC | TGF beta responsive transcription factor Smad3 in nuclear with histone deacetylase |
| Wee1: Cdc2 | kinase Wee1 is negative regulator of Cdc2 (cell cycle progression kinase) |
| Akt1: p27 | Intersection of key anti-apoptotic (Akt) and cell cycle regulatory (p27) signaling nodes. Both targets |
| | invovled in human tumors. |
| p27: Ubiquitin | p27 is key cell cycle regulator; loss is associated with human tumor progression. p27 levels are |
| | controlled by ubiquitination. |
| CDC25C: Cdc2 | Phosphatase/kinase complex; activity leads to cell cycle progression |
| E6: p53 | indicates p53 primed for proteasomal degradation |
| p53: p53 | increased interaction and dimerization of p53 indicates heightened activity of this node |
| ERK-P + VEGF | ERK mitogen-activated protein kinase is activated by signaling through the vascular endothelial growth |
| | factor pathway |

TABLE 2
Description, Genbank identifiers and reporter fusion orientations
for protein-fragment complementation assays

| | | | Reporter | | Reporter |
|---|---|---|---|---|---|
| | | Genbank | fusion | Genbank | fusion |
| PCA Description | Stimulation | Gene 01 | orientation | Gene 02 | orientation |
| BAD: BID | | NM_004322 | N | NM_001196 | C |
| Bcl-xL: Bad | | NM_138578 | C | NM_004322 | N |
| Bcl-xL: BIK | | NM_138578 | N | NM_001197 | N |
| Cdc2: Cdc25A +CPT | 500 nM CPT; 16 hrs | NM_001786 | N | NM_001789 | C |
| Cdc2: CDC25C | | NM_001786 | N | NM_001790 | C |
| Cdc2: CDC25C +CPT | 500 nM CPT; 16 hrs | NM_001786 | N | NM_001790 | C |
| Cdc2: Wee1 | | NM_001786 | N | NM_009516 | N |
| CDC42: PAK4 | | NM_001791 | N | NM_005884 | C |
| Chkl: CDC25A +CPT | 500 nM CPT; 16 hrs | NM_001274 | N | NM_001789 | C |
| Chkl: CDC25C | | NM_001274 | N | NM_001790 | C |
| Chkl: CDC25C +CPT | 500 nM CPT; 16 hrs | NM_001274 | N | NM_001790 | C |
| Cofilin: LIMK2 | | NM_005507 | C | NM_005569 | N |
| CyclinD: Cdk4 | | NM_053056 | N | NM_001791 | C |
| CDC25C: CDC2 | | NM_001274 | N | NM_001786 | N |
| E6: p53 | | AJ388069 | N | NM_000546 | N |
| H-Ras: Raf | | NM_005343 | N | NM_002880 | C |
| Hsp90: CDC37 | | NM_007355 | C | NM_007065 | N |
| Hsp90: Eef2k | | NM_007355 | C | NM_007908 | N |
| MAX: MYC | | NM_002382 | C | NM_002467 | C |
| CDC2: p21 | | NM_001786 | N | NM_000389 | N |
| P27: UbiquitinC | | NM_004064 | N | NM_021009 | N |
| | | | | (CDS | |
| | | | | 69 . . . 296) | |
| p53: Chkl | | NM_000546 | C | NM_001274 | N |
| p53: Chkl +CPT | 500 nM CPT; 16 hrs | NM_000546 | C | NM_001274 | N |
| p53: p53 | | NM_000546 | C | NM_000546 | C |
| p53: p53 +CPT | 500 nM CPT; 16 hrs | NM_000546 | C | NM_000546 | C |
| PAK4: Cofilin | | NM_005884 | C | NM_005507 | C |
| Smad3: HDAC | | NM_005902 | N | NM_004964 | C |

Immunofluorescence Methods

Immunofluorescence was performed on drug-treated cells to assess the post-translational modification status of proteins involved in the pathways of interest. We constructed assays designed to measure the phosphorylation status of key signaling proteins in the absence and presence of a growth factor stimulus. A drug capable of blocking or inhibiting the pathway leading to the signaling protein would in principle cause a decrease in the phosphorylation of that signaling protein in response to the selected growth factor. Such a change in phosphorylation status could be measured by a decrease in fluorescence in the presence of the drug. To exemplify the approach, we studied changes in the phosphorylation status of the protein kinase, ERK (mitogen activated protein kinase) in the MAP kinase pathway that is linked to the angiogenic growth factor, VEGF (vascular endothelial growth factor).

HEK293T cells were seeded at a density of 7,500/well in poly-D-lys coated, blackwalled 96 well plate (Greiner). After 24 hours, cells were transfected with 100 ng/well mVEGFR2 in the pCDNA3.1 expression vector. Forty-eight hours following transfection, the cells were incubated in the absence or presence of indicated drugs for 90 min. The cells were stimulated with 50 ng/ml mVEGF (R & D Systems) during the last 5 min of drug treatment and fixed with 4% formaldehyde in PBS for 15 min. For antibody staining, cells were permeabilized with 0.25% Triton X-100 for 6 min and non-specific staining was blocked by incubating the cells with 3% BSA in PBS for 15 min. Phosphorylated ERK was detected by incubating the fixed cells with rabbit phospho-ERK (T202/Y204)-specific antibodies (Cell Signaling Technologies) followed by Alexa488 conjugated goat anti-rabbit antibody (Molecular Probes). Cell nuclei were stained with Hoechst 33342 (Molecular Probes). A solution of 5% glycerol (in PBS) solution was used to overlay the cells. Fluorescence images were acquired on a Discovery-1 imaging station (Molecular Devices) as described above.

Background fluorescence due to nonspecific binding by the secondary antibody was established with the use of cells that were incubated with BSA/PBS and without primary antibodies.

Fluorescence Image Analysis

To allow quantitation of the drug effects observed by fluorescence microscopy in the PCA and immunofluorescence assays, we applied image analysis algorithms to the images acquired by automated microscopy. A variety of commercial software packages for the analysis of cell-based ‘high content’ assays are suitable for this purpose and are commercially available (Cellomics; GE Medical/Amersham; BectonDickinson/Atto Bioscience; Beckman Coulter/Q3DM; and others). We used publicly-available software (ImageJ API/library (http://rsb.info.nih.gov/ij/, NIH, MD) to analyze the raw images in 16-bit grayscale TIFF format. First, images from the fluorescence channels were normalized using the ImageJ built-in rolling-ball algorithm [S. R. Sternberg, Biomedical image processing. Computer, 16(1), January 1983]. Next a threshold was established to separate the foreground from background. An iterative algorithm based on Particle Analyzer from ImageJ was applied to the thresholded Hoechst channel image (HI) to obtain the total cell count. The nuclear region of a cell (nuclear mask) was also derived from the thresholded HI. The positive particle mask was generated from the thresholded YFP image (YI). To calculate the global background (gBG), a histogram was obtained from the un-thresholded YI and the pixel intensity of the lowest intensity peak was identified as gBG. Masks from different fluorescence channels were overlapped to define the correlated sub-regions of the cell. The mean pixel intensity for all positive particles within each defined sub-region was calculated, resulting in multiple parameters: MT, the mean intensity of the total fluorescence); M1, the mean intensity of the Hoechst defined region); M2, the mean intensity of the WGA-defined region, where used; and M3, the mean intensity of the pixels excluded from the other regions). All means were corrected for the corresponding gBG.

For each set of experiments (assay+drug treatment+treatment time), fluorescent particles from eight images were pooled. For each parameter, an outlier filter was applied to filter out those particles falling outside the range (mean±3SD) of the group. The sample mean or control mean for each parameter was obtained from each filtered group.

MTT Proliferation Assays

Human non-small cell lung carcinoma (A549, ATCC # CCL-185), colon adenocarcinoma (LoVo, ATCC # CCL-229), pancreatic carcinoma (MIA PaCa-2, ATCC # CRL-1420), prostate adenocarcinoma (PC-3, ATCC # CRL-1435), and glioblastoma (U-87 MG, ATCC # HTB-14) cells were acquired from American Type Culture Collection (ATCC, Manassas, Va.). Cells were maintained in various media as follows: A549, LoVo and PC-3 (Ham's F12K medium with 2 mM L-glutamine and 1.5 g/L sodium bicarbonate), MIA PaCa-2 (Dulbecco's modified Eagle's medium with 4 mM L-glutamine and 4.5 g/L glucose), U87-MG (MEM+Earle's BSS). Medium for each cell line was supplemented with 10% FBS and 100 mg/ml Penecillin/Streptomycin. All cells were grown in incubators set at 37° C., 5% CO₂. Thiazolyl Blue Tetrazolium Bromide (MTT) based proliferation assays were performed to assess the anti-proliferative activities of the compounds on these cells. Cells were seeded in 96 well plates at a density of 750 cells/well 24 hours prior to compound treatment. The cells were incubated with varying concentrations of compounds for 120 hours. Compound concentrations range from 0.03 to 100 microM (half log increments) except for alpha-Tomatine (0.001-100 microM, half log increments), Neriifolin (0.0002-100 microM) and Peruvoside (0.01-100 microM). Drug treatment was performed in 5 replicate wells. Background absorbance was established by wells containing medium but no cells. Vehicle (DMSO) only was used as control. MTT (Sigma-Aldrich, St. Louis, Mo.) was added to each well at a final concentration of 0.5 mg/ml. Following a 2 hour incubation at 37° C., medium in the wells was replaced with 0.15 ml DMSO. The plates were agitated for 15 min using a microtiter plate shaker. Absorbance at 560 nM was measured using SpectraMax Plus (Molecular Devices). Mean absorbance values were calculated from 5 replicate wells of each drug treatment following subtraction of background absorbance from blank samples and plotted as a percentage of control.

TABLE 3
Results of screening known drugs to identify new activities

| | | | | | | | | | Tumor Cell | Anti- | |
|---|---|---|---|---|---|---|---|---|---|---|---|
| | | | | | Patent | Generics | | | (Selected Tumor | proliferative | Original |
| Compound Name | Synonyms | Drug target | Developer | Marketing Status | Eaclusivity | (Y/N) | Pathway Activity | Assay activity | Model) | IC-50 (uM) | Indication |
| Bepridil | Bepadin | Inhibitor of | Medpointe | FDA Approved | Expired | No | invasion/ | CDC42/PAK4 | NSCLC | 4.74 | Angina |
| | Vascor | Ca(2 + YNa + | | | | | metastasis | | Colon | 3.68 | |
| | | exchanger/ | | | | | | | Pancreatic | 1.58 | |
| | | Calcium channel | | | | | | | Prostate | 8.69 | |
| | | blocker | | | | | | | Glioblastoma | 5.75 | |
| Cinnarizine | Stugeron | Calcium channel | Jannsen | Approved | None | No | apoptosis | PAK4/CFL | NSCLC | 5.71 | Asthma, |
| | | blocker | | outside US | | | | CDC42/PAK4, | Colon | 7.14 | Migraine, |
| | | | | | | | | | Pancreatic | 11.11 | antiemetic |
| | | | | | | | | | Prostate | 10.5 | |
| | | | | | | | | | Glioblastoma | 13.5 | |
| Peruvoside | | Na+/K+ ATPase | | Never marketed | None | No | cell cycle | Wee1/Cdc2 | NSCLC | 0.004 | Congestive heart |
| | | | | in U.S | | | | | Colon | 0.009 | failure, Clinical |
| | | | | | | | | | Pancreatic | 0.003 | trial (Verga, ′75, |
| | | | | | | | | | Prostate | 0.005 | Dalal ′72, Bhatia, |
| | | | | | | | | | Glioblastoma | 0.002 | insufficiency) |
| Sanguinarine | | Anti-bacterial | | Never marketed | None | No | proliferation | Ras/Raf | NSCLC | 0.78 | Periodontitis, |
| | | | | in U.S. | | | | | Colon | 0.81 | Dental |
| | | | | | | | | | Pancreatic | 0.18 | Hygeine:oral |
| | | | | | | | | | Prostate | 0.33 | rinse, toothpaste |
| | | | | | | | | | Glioblastoma | 0.34 | |
| Terfenadine | Seldane | anti-histamine | Hoechst | Withdrawn | Expired | No | invasion/ | CDC42/PAK4 | NSCLC | 0.55 | Allergies, |
| | | | | | | | metastasis | | Colon | 0.99 | Histamine HI |
| | | | | | | | | | Pancreatic | 0.35 | Raceptor |
| | | | | | | | | | Prostate | 1.32 | Antagonist |
| | | | | | | | | | Glioblastoma | 0.38 | |
| Desipramine | Norpramine | increases brain | Aventis | FDA Approved | Expired | Yes | proliferation | Ras/Raf | NSCLC | 11.88 | Depression |
| | Pertofrane | serotonin and | | | | | | | Colon | 10.6 | |
| | | norepinephrine, | | | | | | | Pancreatic | 1.93 | |
| | | facilitates | | | | | | | Prostate | 14.6 | |
| | | monoamine | | | | | | | Glioblastoma | 6.90 | |
| | | neurotransmission, | | | | | | | | | |
| | | and increases IL- | | | | | | | | | |
| Fenofibrate | Tricor | PPAR alpha | Abbott | FDA Approved | Expired | Yes | apoptosis | Cofilin1/LIMK2, | NSCLC | 15.85 | Hypercholesterolemia; |
| | Lipidil | | | | | | | BCL-xL/BIK, | Colon | 13.2 | hypertriglyceridemia |
| | | | | | | | | p27/Ub, | Pancreatic | 8.4 | |
| | | | | | | | | AKT1/p27, | Prostate | 18 | |
| | | | | | | | | cyclinD1/CDK4, | Glioblastoma | 40 | |
| | | | | | | | | phosphoo-ERK | | | |
| | | | | | | | | (+VEGF) | | | |
| Droperidol | Inapsine | | Akom | FDA Approved | Expired | Yes | invasion/ | PAK4/CFL | NSCLC | 24.3 | Adjunct with |
| | | | | | | | metastasis | | Colon | 25.8 | Anesthesia; |
| | | | | | | | | | Pancreatic | 8.8 | antimetic |
| | | | | | | | | | Prostate | 7.68 | antipsychotic |
| | | | | | | | | | Glioblastoma | 24.6 | |
| Promazine | Sparine | | Wyeth | Discontinude | Expired | No | proliferation | Ras/Raf | NSCLC | 13.9 | Sedative |
| | Prozine | | | | | | | | Colon | 13.9 | Antipsychotic |
| | | | | | | | | | Pancreatic | 5.13 | |
| | | | | | | | | | Prostate | 13.9 | |
| | | | | | | | | | Glioblastoma | 16.4 | |
| Suloctidil | Sulocton | | 7 | Approved | None | No | invasion/ | PAK4/CFL, | NSCLC | 2.23 | dementia |
| | | | | outside US | | | metastasis | CDC42/PAK4 | Colon | 1.13 | antithrombotic |
| | | | | | | | | | Pancreatic | 1.93 | |
| | | | | | | | | | Prostate | 1.85 | |
| | | | | | | | | | Glioblastoma | 1.12 | |
| Metergoline | none | | | Never marketed | None | No | invasion/ | Ras/Raf: | NSCLC | 5.4 | Hyperprolactinaemia. |
| | | | | in U.S. | | | metastasis | CDC42/PAK4 | Colon | 6.97 | seratonin |
| | | | | | | | | | Pancreatic | 3.35 | receptor blocker |
| | | | | | | | | | Prostate | 6.19 | |
| | | | | | | | | | Glioblastoma | 4.6 | |
| Niclosamide | Niclocide | | Bayer | Discontinued | Expired | No | apoptosis | HSP90/CDC37, | NSCLC | 0.66 | Antihelmintic |
| | | | | | | | | HSP90/Eef2k, | Colon | 0.45 | |
| | | | | | | | | Cofilin/LIMK2, | Pancreatic | 0.59 | |
| | | | | | | | | PIN1/JUN, | Prostate | 1.33 | |
| | | | | | | | | HDAC/SMAD3, | Glioblastoma | 0.81 | |
| | | | | | | | | MYC/MAX | | | |
| Neri$$olin | | Na+/K+ ATPase | NCl | Never marketed | None | No | invasion/ | PAK4/CFL, | NSCLC | 0.005 | Congestive heart |
| | | | | in U.S. | | | metastasis | CDC42/PAK4 | Colon | 0.018 | failure |
| | | | | | | | | | Pancreatic | 0.004 | |
| | | | | | | | | | Prostate | 0.011 | |
| | | | | | | | | | Glioblastoma | | |
| Flunarizine | Sibelium | Calcium channel | Jannsen | Approved | None | No | invasion/ | PAK4/CFL, | NSCLC | 18.23 | prophylaxis of |
| | | blocker, Na+ | | outside US | | | metastasis | CDC42/PAK4 | Colon | | migraine |
| | | Channel | | | | | | | Pancreatic | 22.53 | |
| | | antagonist | | | | | | | Prostate | | |
| | | | | | | | | | Glioblastoma | | |
| Tomatine | Lycopersicin | | | Never marketed | None | No | invasion/ | Cdc42/PAK4 | NSCLC | 0.092 | Adjuvant |
| | | | | in U.S. | | | metastasis | | Colon | 0.097 | |
| | | | | | | | | | Pancreatic | 0.131 | |
| | | | | | | | | | Prostate | 0.09 | |
| | | | | | | | | | Glioblastoma | | |
| Albendazole | Albenza | Helminths, | GSK | FDA Approved | Expired | No | invasion/ | CDC42/PAK4 | NSCLC | 0.24 | Antihelmintic |
| | | parasites | | | | | metastasis | | Colon | 0.14 | |
| | | | | | | | | | Pancreatic | 0.18 | |
| | | | | | | | | | Prostate | 0.3 | |
| | | | | | | | | | Glioblastoma | | |
| Mebendazole | Vermox | | McNeil | FDA Approved | Expired | Yes | invasion/ | PAK4/CFL | NSCLC | 0.18 | Antihelmintic |
| | | | | | | | metastasis | | Colon | 0.07 | |
| | | | | | | | | | Pancreatic | 0.13 | |
| | | | | | | | | | Prostate | 0.27 | |
| | | | | | | | | | Glioblastoma | | |
| Clomiphene | Clomid | | Aventis | FDA Approved | Expired | Yes | proliferation | Ras/Raf | NSCLC | 8.35 | Olulatory |
| | Milophene | | | | | | | | Colon | | disfunction |
| | Serophene | | | | | | | | Pancreatic | 3.0 | |
| | | | | | | | | | Prostate | | |
| | | | | | | | | | Glioblastoma | | |
| Dichlorophene | | | | Veterinary | None | No | proliferation | Ras/Raf | NSCLC | 9.83 | Antihelminthic; |
| | | | | | | | | | Colon | | antiprotozoan |
| | | | | | | | | | Pancreatic | 25.8 | |
| | | | | | | | | | Prostate | | |
| | | | | | | | | | Glioblastoma | | |
| Meclocycline | Meclan | | J&J | Discontinued | Expired | no | invasion/ | CDC42/PAK4 | NSCLC | 18.76 | antibiotic |
| | | | | | | | metastasis | | Colon | | |
| | | | | | | | | | Pancreatic | 68.21 | |
| | | | | | | | | | Prostate | | |
| | | | | | | | | | Glioblastoma | | |
| Atorvastatin | Lipitor | | Pfizer | FDA Approved | Protected | No | invasion/ | CFL1/LIMK2, | NSCLC | >40 | Hypercholesteremia |
| | | | | | | | metastasis | CFL1/PAK4 | Colon | 16.95 | |
| | | | | | | | | | Pancreatic | 6.09 | |
| | | | | | | | | | Prostate | 10.6 | |
| | | | | | | | | | Glioblastoma | 4.6 | |
| beta-Lapachone | | | NCl | Never marketed | None | No | apoptosis | EGFR/Grb2, | NSCLC | 0.23 | sepsis: has |
| | | | | in U.S. | | | | p53/E6, | Colon | 1.4 | known anti- |
| | | | | | | | | CDC25C/Chk1 | Pancreatic | 0377 | cancer activity |
| | | | | | | | | | Prostate | 0.63 | |
| | | | | | | | | | Glioblastoma | 0.86 | |
| Sertraline | Zoloft | | Pfizer | FDA Approved | Protected | No | apoptosis | BAD/BID, | NSCLC | 1.22 | depression |
| | | | | | | | | CDC2/CDC25C, | Colon | 2.79 | |
| | | | | | | | | EGFR/Grb2, | Pancreatic | 0.87 | |
| | | | | | | | | Eef2K/hsp90, | Prostate | 4.46 | |
| | | | | | | | | p53/p53, | Glioblastoma | 7.4 | |
| | | | | | | | | MAX/MYC | | | |

Results

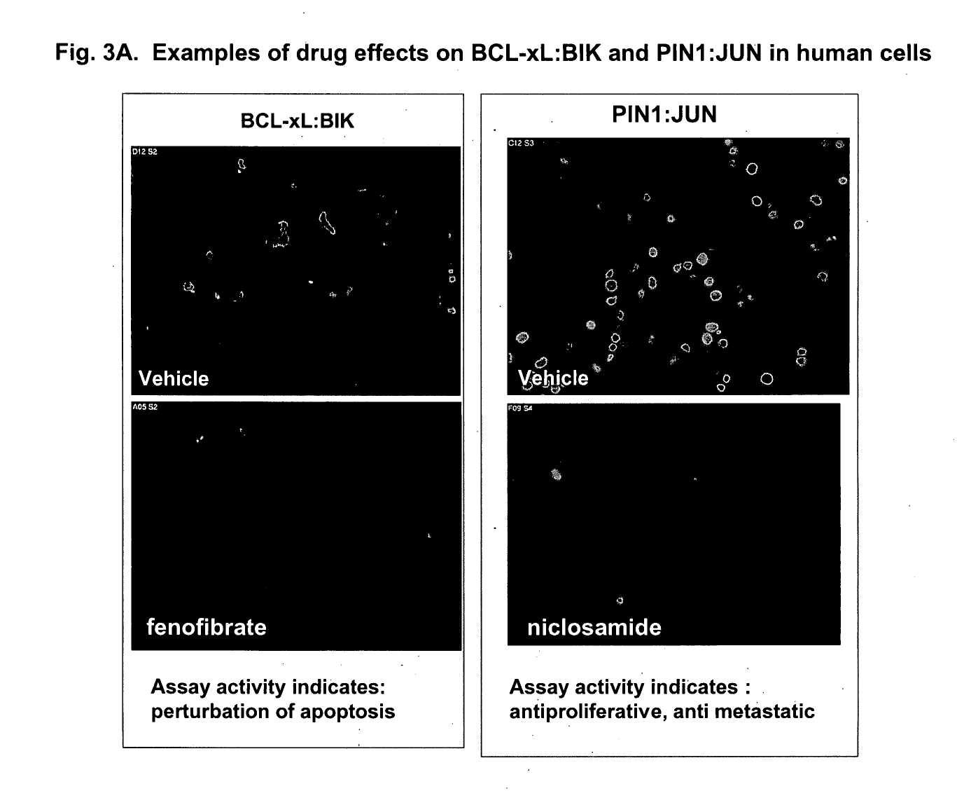

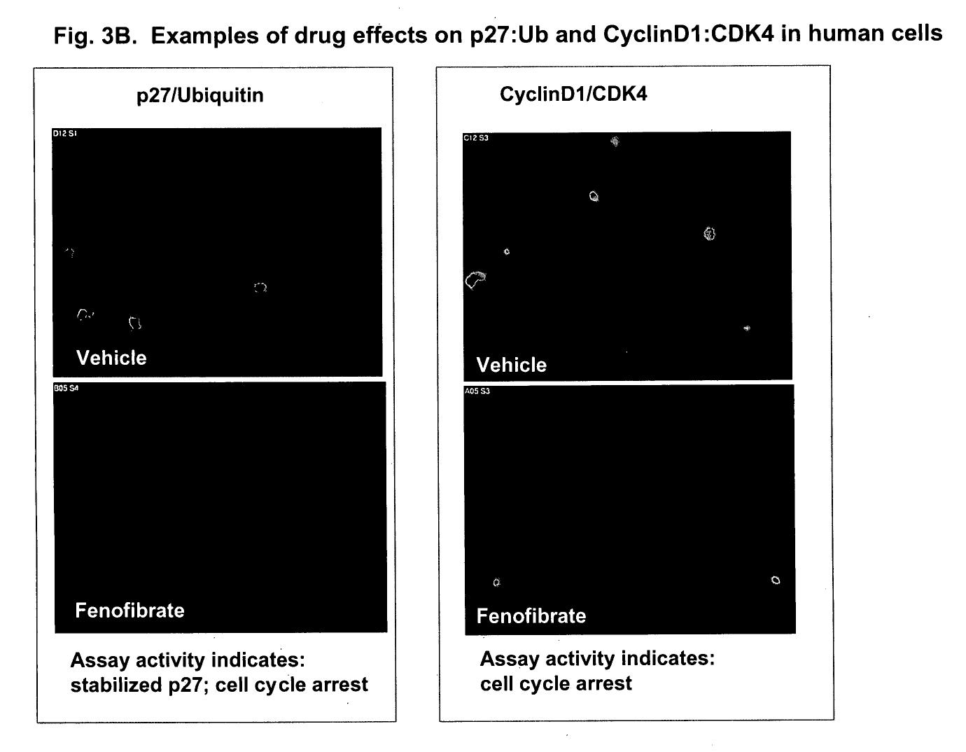

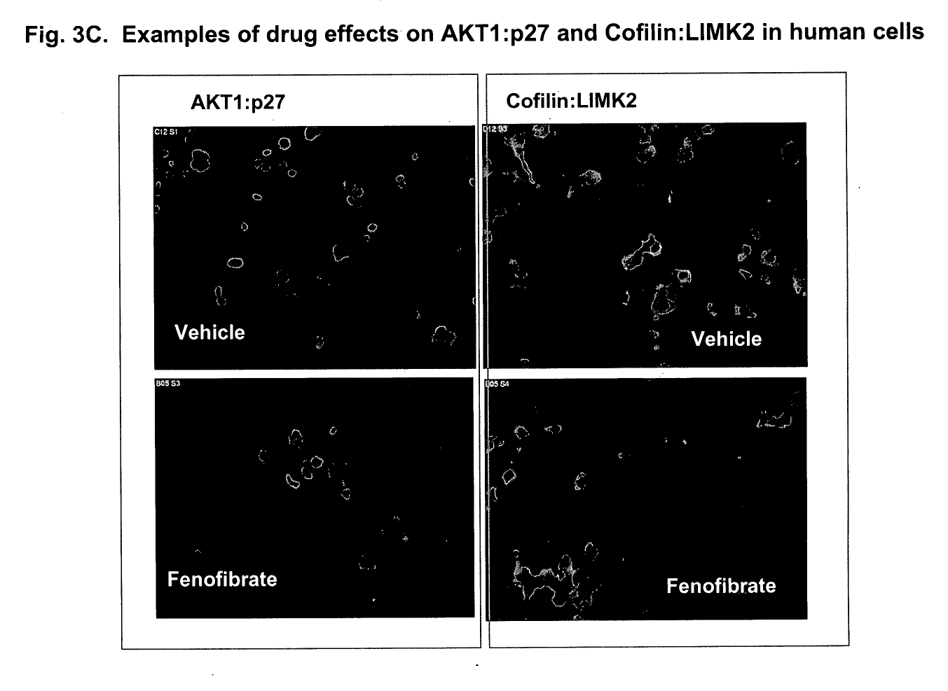

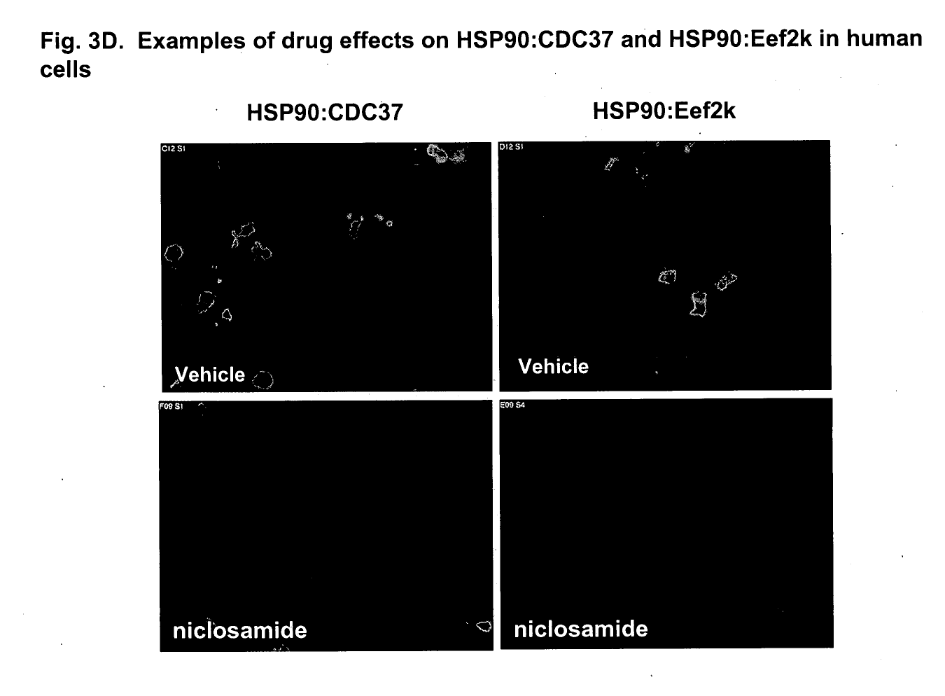

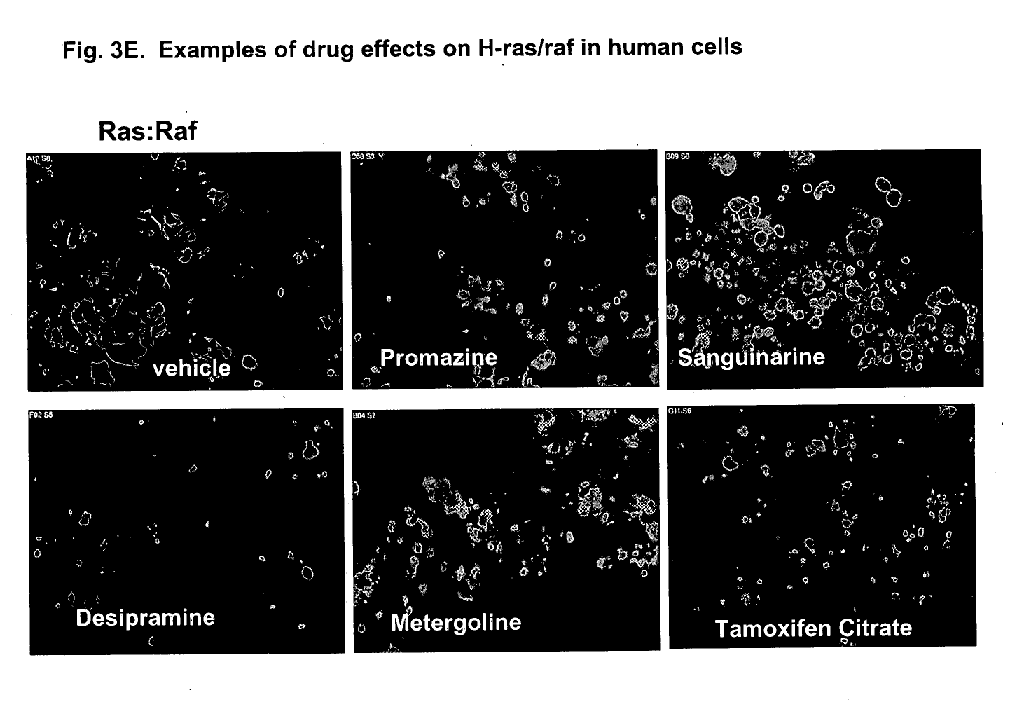

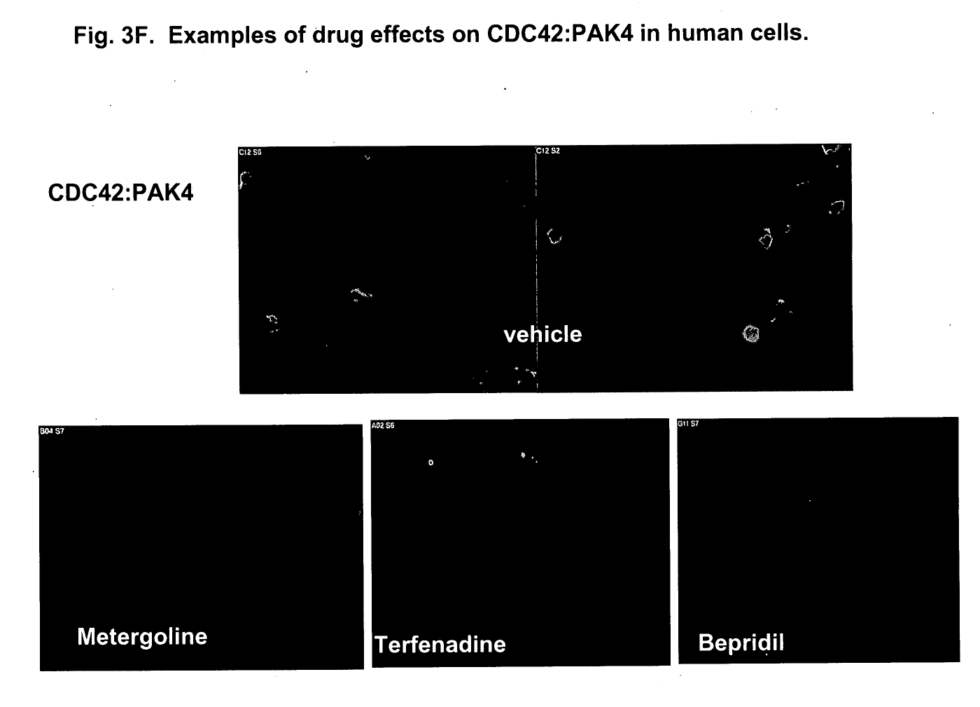

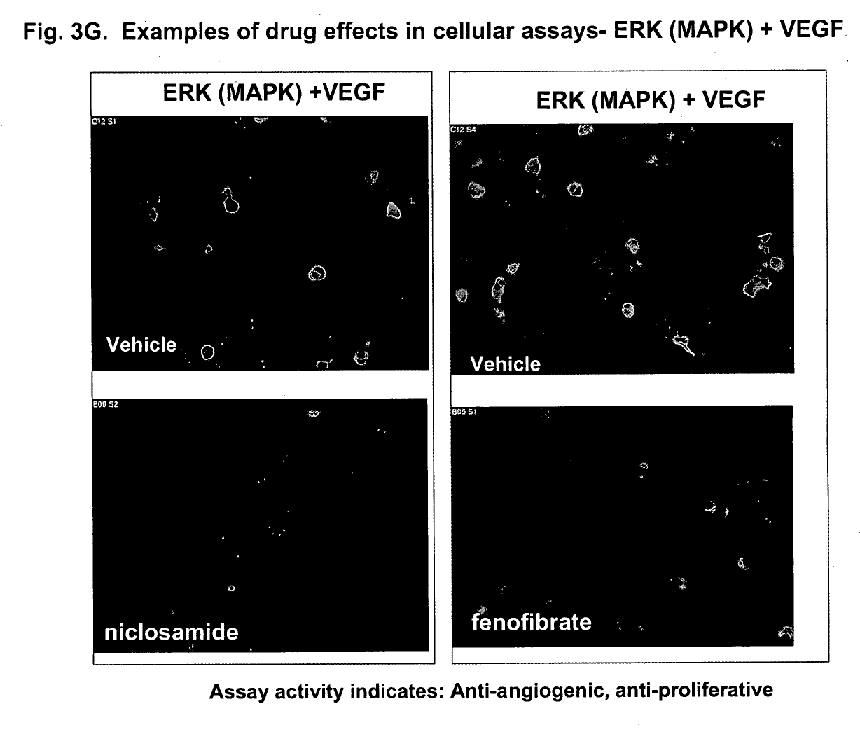

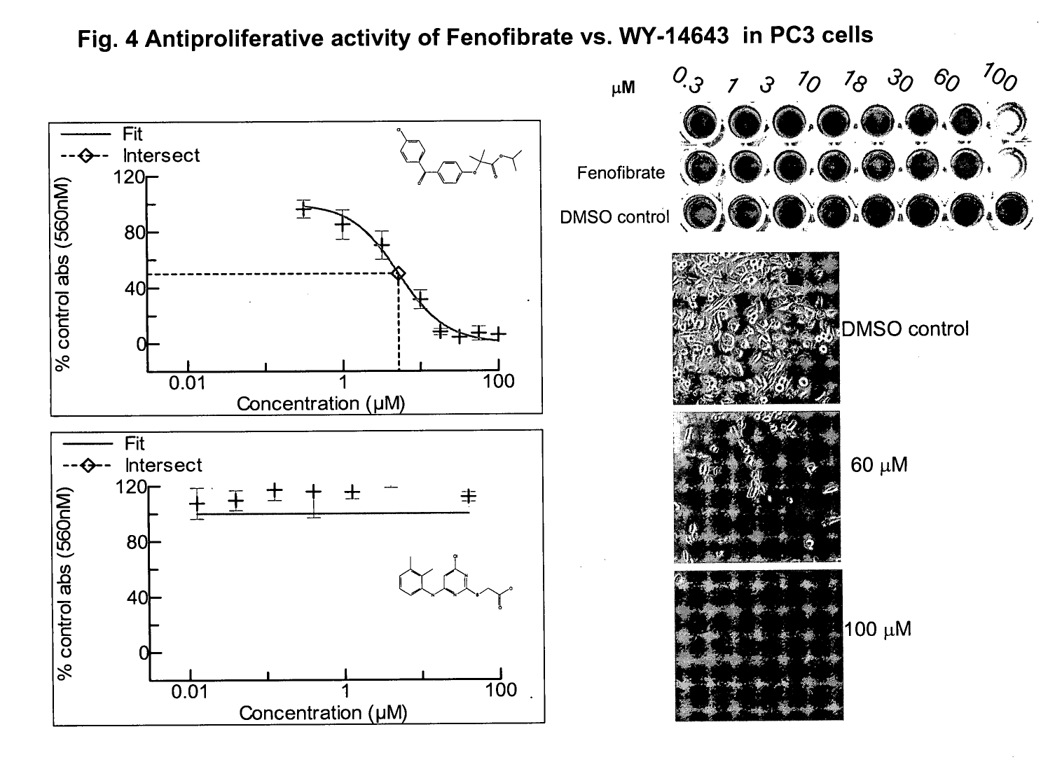

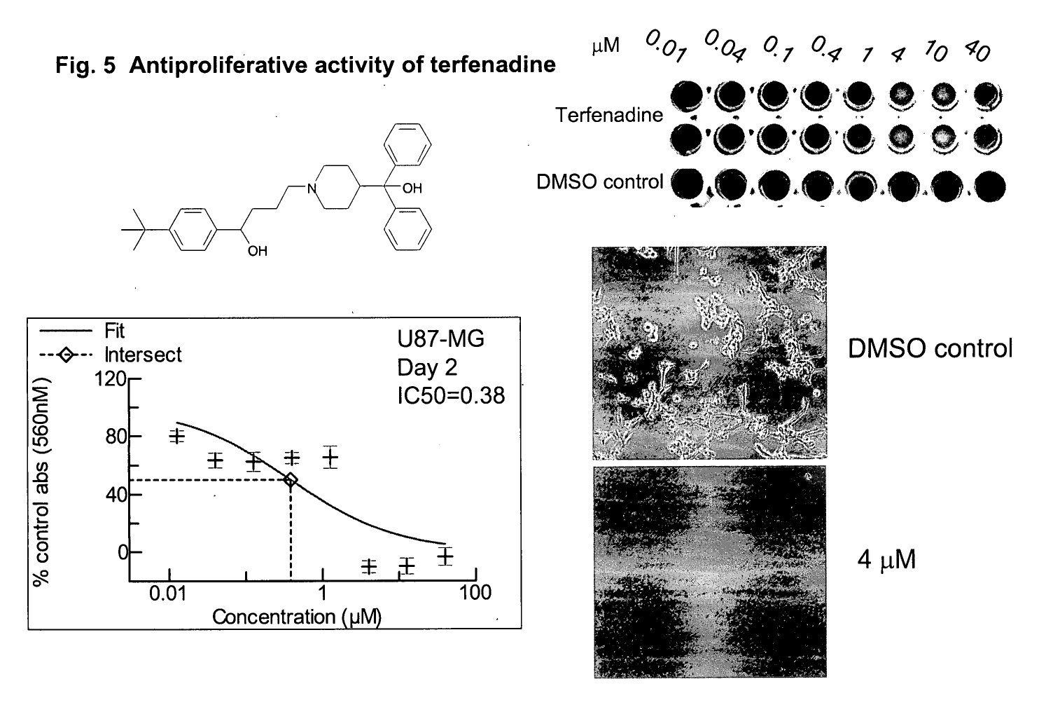

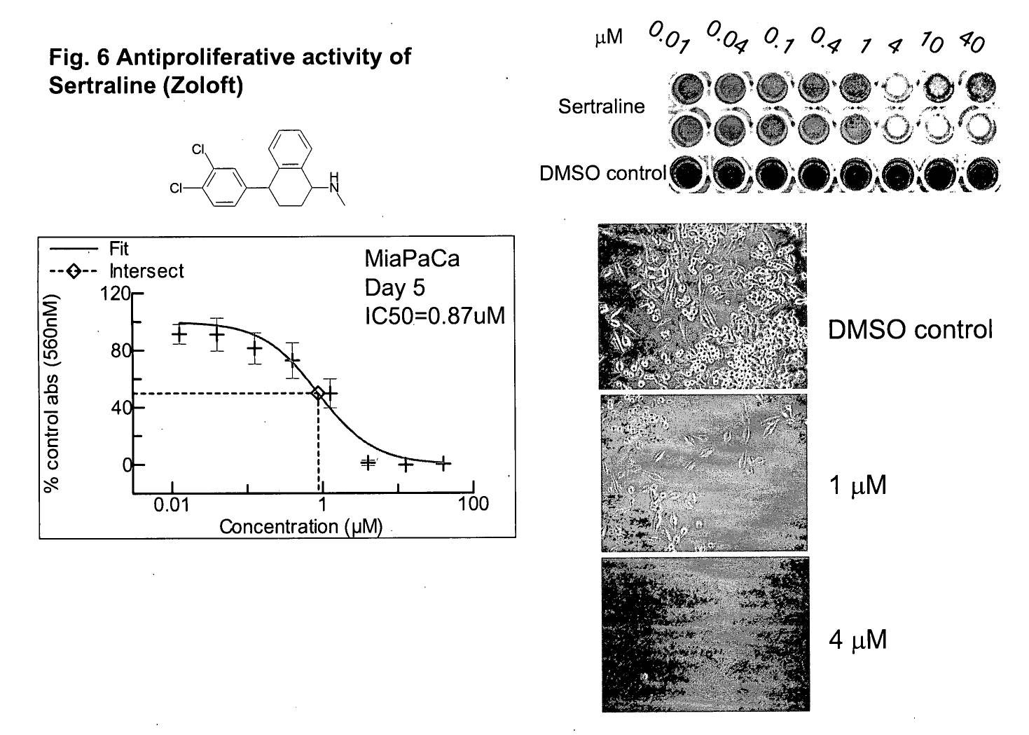

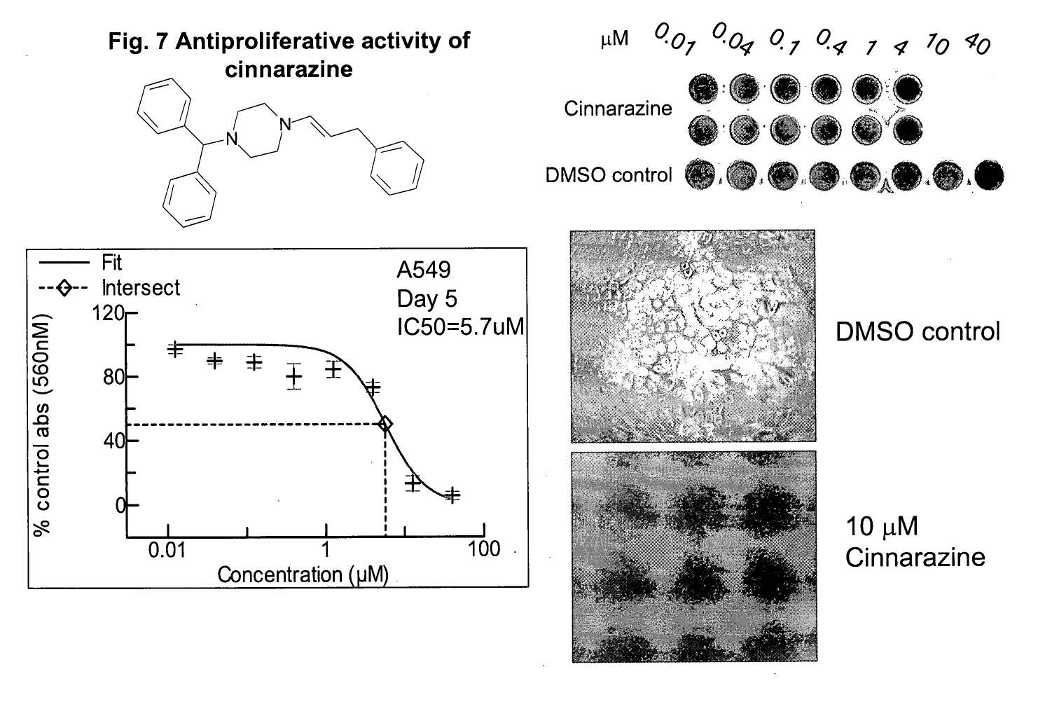

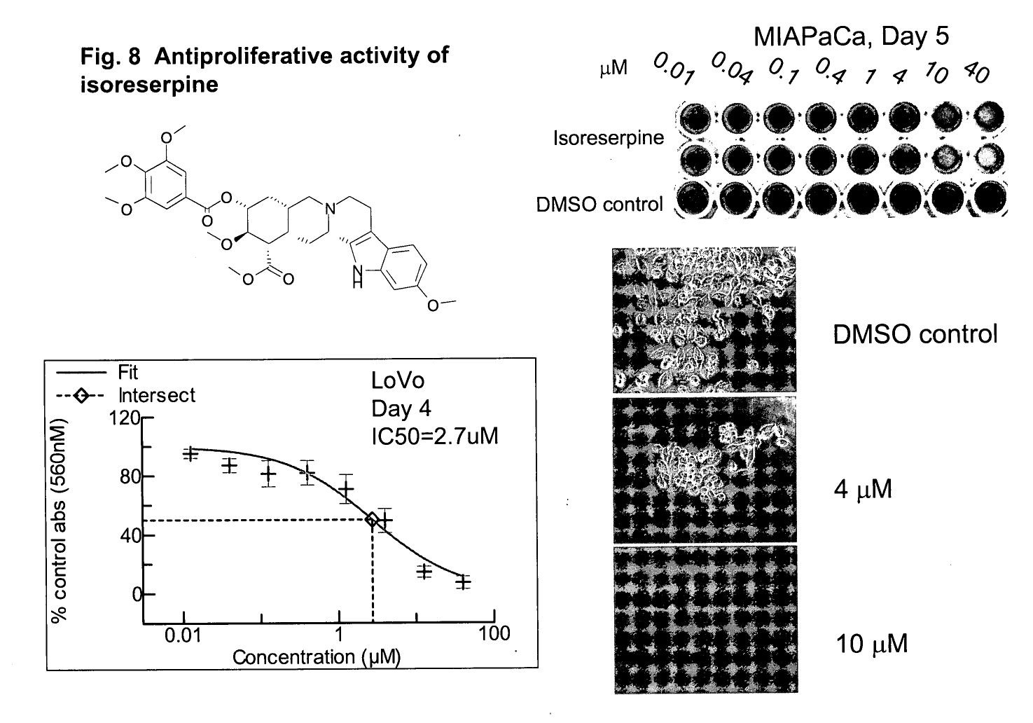

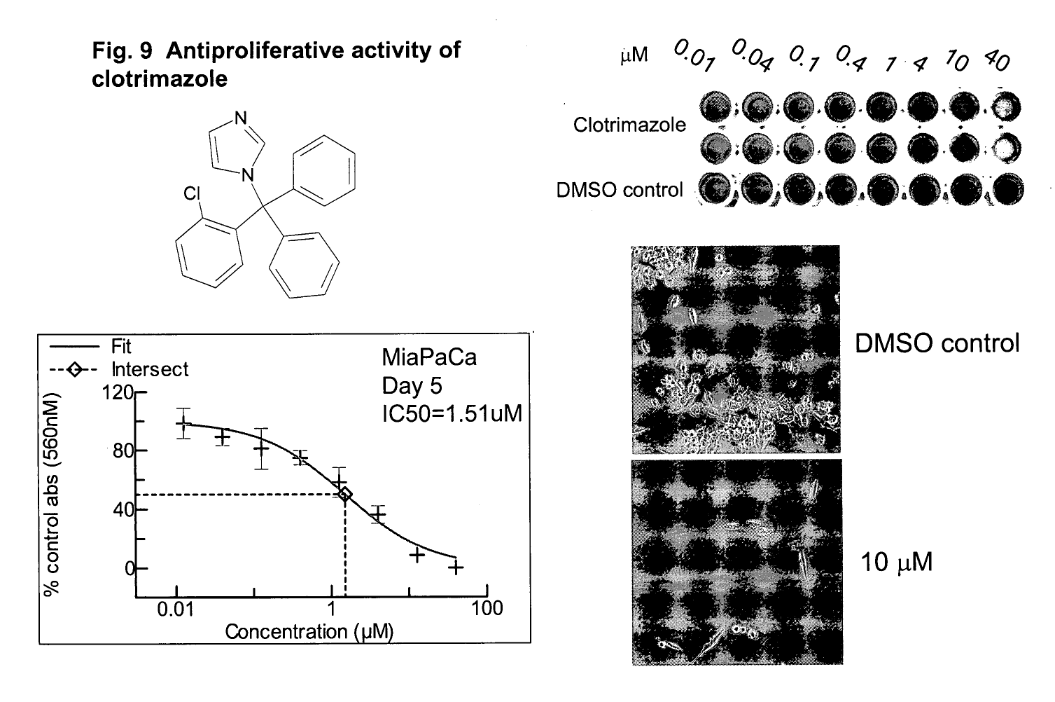

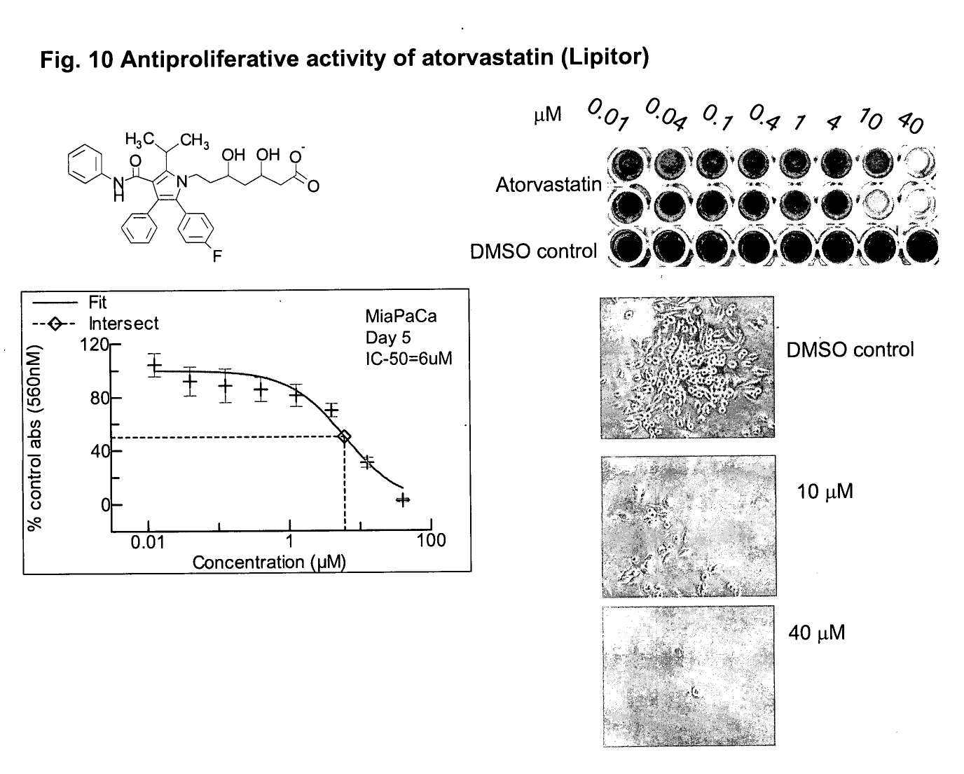

Photomicrographs showing specific assay results are shown in FIG. 3A-3G. Antiproliferative activities of selected drugs are shown in FIGS. 4-10. A summary of drugs that ‘hit’ specific pathways is shown in Table 3, together with the assay activities and the activity of each drug on the proliferation of human tumor cell lines as assessed in the MTT assay. The IC50 for proliferation (concentration of each drug that inhibits proliferation by 50%) is shown in Table 3 for each tumor cell line that was tested.

A variety of drug mechanisms of action may underlie the activities seen here. For example, the amount of a protein complex may vary as a result of increased formation, decreased formation, or a decrease in stability of one or more of the components. Biochemical mechanisms underlying the changes include changes in post-translational modification status of one or more proteins in the complex; inhibition of chaperone function; proteasome inhibition; pathway inhibition in the presence of a stimulus; direct inhibition of a protein-protein interaction; and other potential mechanisms. Any of these mechanisms may result in a change in the amount, subcellular location, or post-translational modification status of the cognate complexes, as measured here.

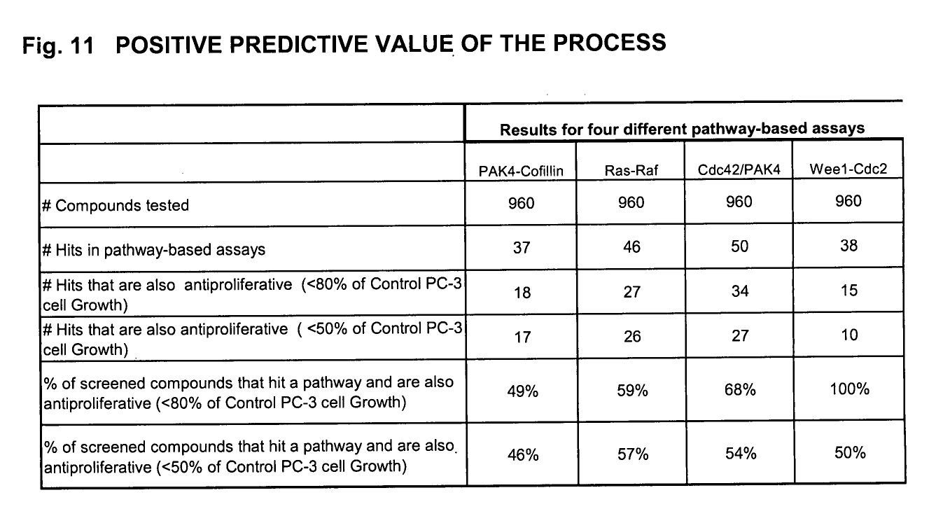

There are several features of our results that are particularly interesting. First, the entire screening process is extremely fast: the identification of drugs with promising antiproliferative activity could be completed in two weeks following assay construction. Second, the pathway-based screens we constructed were remarkable indicators of antiproliferative activity with individual assays giving positive predictive values that in most cases exceeded 50% (FIG. 11). Combinations of assays gave positive predictive values of 70-80%. Third, many of the drugs hit two or more cancer pathways. This supports our notions of the interconnectivity of cellular networks and also shows that even well-characterized drugs have significant and surprising ‘off-pathway’ effects. Fourth, the number of drugs found to have anti-proliferative activity in this proof-of-principle study was 23/960 or over 2%, which is a substantial rate in terms of reindicating drugs for new therapeutic uses. This also suggests that the overall strategy presented here, if applied to a broad range of disease pathways, will provide a powerful strategy for identifying medicines that can be fast-tracked for the treatment of human disease. Since these drugs have well-characterized safety profiles they can quickly be advanced into clinical trials for the new therapeutic indications.

Remarkably, in most cases, the doses of drugs that inhibited proliferation of tumor cells were in the low-micromolar range and in fact well within the range of plasma levels for that drug (where documented in the literature).

It will be appreciated by one skilled in the art that the exact sentinels (proteins, and protein-protein interactions) to be used for this strategy will depend upon the disease of interest and that the invention is not limited to the particular pathways, proteins, or sentinels provided herein or to a particular mechanism by which a drug affects that pathway. For example, to identify anti-proliferative agents, we used pathways that contribute to the cancer phenotype. However, the methodology applied in the current invention is not limited to the identification of anti-cancer activities of drugs. For other diseases we are probing pathways characteristic of those disorders: e.g. for diabetes, we are studying pathways involved in glucose transport, glycogenesis, insulin receptor regulation and insulin signaling pathways. For bone disease, we are using pathways that are involved in bone remodeling and the differential activity of osteoclasts and osteoblasts. For neurological disorders we are using pathways that are downstream of the dopamine receptor and the serotonin receptor.

The invention can also be applied to cell types other than mammalian cells. For example, the invention can be applied to the discovery of antibiotic agents, antifungal agents, antiviral agents, and other infectious diseases. In these cases the cell of interest (bacterial, fungal, etc.) can be used to construct the assays, in conjunction with the pathways/proteins of interest for the disease in question. For example, an agent that disrupts a pathway that is key for the survival of a bacterial cell may be a useful antibiotic agent. In the case of antiviral agents, mammalian cells can be used for the readout and viral/host interacting proteins can be used to construct the assays. For example, an agent that disrupts a host protein receptor or a viral/host protein-protein interaction—either directly or indirectly—may have utility in the prevention or treatment of viral infection.

Many ideas for new assays can be gleaned from the biochemical literature and applied, in conjunction with the methods provided herein, to identifying new indications for known drugs. The genes to be used in the assays may code either for known or for novel interacting proteins. The interacting proteins may be selected by one or methods that include bait-versus-library screening; pairwise (gene by gene) interaction mapping; and/or prior knowledge or a hypothesis regarding a pathway or an interacting protein pair. In addition, novel pathways that are useful for drug discovery can be identified empirically by constructing assays for novel protein protein interactions; determining if these are responsive to agents known to affect the pathway(s) of interest; and using the resulting novel assays to screen for known drugs as well as for new chemical entities with desired activities.

### Methods Suitable for Use with the Screening System

The screening system presented here can be used in several different modes including high-throughput screens (HTS) and high-content screens (HCS). In the case of purely quantitative assays (HTS), the signal generated in the assay is quantified with a microtiter fluorescence plate reader, flow cytometer, fluorimeter, microfluidic device, or similar devices. The intensity is a measure of the quantity of the protein-protein complexes formed and allows for the detection of changes in protein-protein complex formation in live cells in response to agonists, antagonists and inhibitors. In the case of high-content assays (HCS), cells are imaged by automated microscopy, confocal, laser-based, or other suitable high-resolution imaging systems. The total fluorescence per cell as well as the sub-cellular location of the signal (membrane, cytosol, nucleus, endosomes, etc.) can be detected. Cell fixation offers advantages over live cell assays for purposes of laboratory automation, since entire assay plates can be fixed at a specific time-point after cell treatment, loaded into a plate stacker or carousel, and read at a later time.

The choice of HTS or HCS formats is determined by the biology and biochemistry of the signaling event and the functions of the proteins being screened. It will be understood by a person skilled in the art that the HTS and HCS assays that are the subject of the present invention can be performed in conjunction with any instrument that is suitable for detection of the signal that is generated by the chosen reporter.

In addition to the use of live (fixed or unfixed) cells, cell lysates can be prepared following drug treatment and can be used in the present invention. Finally, for specific protein-protein interactions of interest, in vitro assays can be constructed using the methods described herein and used to further study the mechanism of action of any assay hits; to facilitate studies of structure-activity relationships; and to enable de novo discovery of new chemical entities with desired activities.

Methods for the Detection or Measurement of Protein-Protein Interactions

PCA represents a preferred embodiment of the invention. PCA enables the detection and quantitation of the amount and/or subcellular location of protein-protein complexes in living cells. With PCA, proteins are expressed as fusions to engineered polypeptide fragments, where the polypeptide fragments themselves (a) are not fluorescent or luminescent moieties; (b) are not naturally-occurring; and (c) are generated by fragmentation of a reporter. Michnick et al. (U.S. Pat. No. 6,270,964) taught that any reporter protein of interest can be used for PCA, including any of the reporters described in Table 4. Thus, reporters suitable for PCA include, but are not limited to, any of a number of monomeric or multimeric enzymes; and fluorescent, luminescent, or phosphorescent proteins. Small monomeric proteins are preferred for PCA, including monomeric enzymes and monomeric fluorescent proteins, resulting in small (150 amino acid) fragments. Since any reporter protein can be fragmented using the principles established by Michnick et al., assays can be tailored to the particular demands of the cell type, target, signaling process, and instrumentation of choice. Finally, the ability to choose among a wide range of reporter fragments enables the construction of fluorescent, luminescent, phosphorescent, or otherwise detectable signals; and the choice of high-content or high-throughput assay formats.

It will be apparent to one skilled in the art that the choice of expression vector depends on the cell type for assay construction, whether bacterial, yeast, mammalian, or other cell type; the desired expression level; the choice of transient versus stable transfection; and other typical molecular and cell biology considerations. A wide variety of other useful elements can be incorporated into appropriate expression vectors, including but not limited to epitope tags, antibiotic resistance elements, and peptide or polypeptide tags allowing subcellular targeting of the assays to different subcellular compartments (e.g. A Chiesa et al., Recombinant aequorin and green fluorescent protein as valuable tools in the study of cell signaling). The incorporation of a different antibiotic resistance marker into each of the two complementary constructs would allow for the generation of stable cell lines through double antibiotic selection pressure, whereas subcellular targeting elements would allow for the creation of assays for pathway events that occur within a particular subcellular compartment, such as the mitochondria, Golgi, nucleus, or other compartments.

A variety of standard or novel expression vectors can be chosen based on the cell type and desired expression level; such vectors and their characteristics will be well known to one skilled in the art and include plasmid, retroviral, and adenoviral expression systems. In addition, there is a wide range of suitable promoters including constitutive and inducible reporters that can be used in vector construction. If an inducible promoter is used, the signal generated in the assay will be dependent upon activation of an event that turns on the transcription of the genes encoded by the PCA constructs.

The general characteristics of reporters suitable for PCA have previously been described (References incorporated herein). A preferred embodiment of the present invention involves cell-based assays generating a fluorescent or luminescent signal are particularly useful. Examples of reporters that can be used in the present invention have been provided in the References. It will be appreciate by one skilled in the art that the choice of reporter is not limited. Rather, it will be based on the desired assay characteristics, format, cell type, spectral properties, expression, time-course and other assay specifications. For any reporter of interest various useful pairs of fragments can be created, for example using the methods taught in U.S. Pat. No. 6,270,964 and the References incorporated herein, and then engineered in order to generate fragments that produce a brighter signal or a specific color readout upon fragment reassembly. It will be obvious to one skilled in the art that various techniques of genetic engineering can be used to create useful fragments and fragment variants of any of the reporters that are the subject of this invention.

It will be appreciated by a person skilled in the art that the ability to select from among a wide variety of reporters makes the invention particularly useful for drug discovery on a large scale. In particular, reporters can be selected that emit light of a specific wavelength and intensity that may be suitable for a range of protein expression levels, cell types, and detection modes. The flexibility is an important feature of the invention because of the wide range of signaling events, or biochemical processes, that may be linked to drug activity. For some biochemical events, activation of a pathway—for example, by the binding of an agonist—will lead to an increase in the association of a receptor and a cognate binding protein, or of two elements ‘downstream’ in the pathway, such as a kinase and its substrate. An increase in the association of the two proteins that form the PCA pair leads to an increase in the signal generated by the reassembled reporter fragments. In that case, a high-throughput assay format can be used to measure the fluorescent signal that is proportional to the amount of the complex of interest. For quantitative assays, where the readout is an increase or decrease in signal intensity, any of the reporters discussed in the present invention can be used and each reporter has various pros and cons that are well understood by those skilled in the art of cell biology. Enzymes—for which the catalytic reaction generates a fluorescent, phosphorescent, luminescent or other optically detectable signal—may be best suited for purely quantitative assays. Upon fragment complementation, the reconstituted enzyme acts upon a substrate to generate a fluorescent or luminescent product, which accumulates while the reporter is active. Since product accumulates, a high signal-to-noise can be generated upon fragment complementation. Such assays are particularly amenable to scale-up to 384-well or 1536-well formats and beyond, and are compatible with standard and ultra high-throughput laboratory automation.

Preferred reporters for the present invention include but are not limited to a beta-lactamase PCA or a luciferase PCA such as with a firefly luciferase or Renilla luciferase. Each of these enzymes has been successfully used as a cell-based reporter in mammalian systems (S Baumik & SS Gambhir, 2002, Optical imaging of renilla luciferase reporter gene expression in living mice, Proc. Natl. Acad. Sci., USA 2002, 99(1): 377-382; Lorenz et al., 1991, Isolation and expression of a cDNA encoding renilla reniformis luciferase, Proc. Natl. Acad. Sci. USA 88: 4438-4442; G. Zlokarnik et al., 1998, Quantitation of transcription and clonal selection of single living cells with beta-lactamase as reporter, Science 279: 84-88). As an example of the construction of a PCA, beta-lactamase PCAs have been constructed with cell-permeable substrates that generate a high signal to background upon cleavage (A Galarneau et al., 2000, Nature Biotechnol. 20: 619-622). The beta-lactamase PCA is a sensitive and quantitative assay suitable for HTS. This PCA has been used with CCF2/AM, a green fluorescent molecule which becomes blue upon cleavage of the beta-lactam ring by beta-lactamase; the blue-green ratio is therefore a measure of the activity of beta-lactamase which is reconstituted upon protein-fragment complementation. Luciferase PCAs can also be used with cell-permeable substrates to generate HTS assays suitable for the present invention (e.g. R Paulmurugan et al., 2002, Noninvasive imaging of protein-protein interactions in living subjects by using reporter protein complementation and reconstitution strategies, Proc. Natl. Acad. Sci. USA 99: 15608-15613). With suitable modifications, any of these PCAs can also be used in vivo or in vitro for the present invention. It will be apparent to one skilled in the art that PCAs based on inherently fluorescent, phosphorescent or bioluminescent proteins can be read either in high-content formats or in high-throughput formats. These PCAs have the advantage of not requiring the addition of substrate; however, the signal generated is usually lower than that generated by an enzymatic reporter.

Calcium-sensitive photoproteins would be useful as PCAs for such assays. These could be based on fragments of aequorin, obelin; or any other calcium-sensitive protein (e.g. MD Ungrin et al., 1999, An automated aequorin luminescence-based functional calcium assay for G-protein-coupled receptors, Anal Biochem. 272: 34-42; Rizzuto et al., 1992, Rapid changes of mitochondrial calcium revealed by specifically targeted recombinant aequorin, Nature 358 (6384): 325-327; Campbell et al., 1988, Formation of the calcium activated photoprotein obelin from apo-obelin and mRNA in human neutrophils, Biochem J. 252 (1):143-149). Aequorin, a calcium-sensitive photoprotein derived from the jellyfish Aequorea victoria, is composed of an apoprotein (molecular mass ˜21 kDa) and a hydrophobic prosthetic group, coelenterazine. Calcium binding to the protein causes the rupture of the covalent link between the apoprotein and the coelenterazine, releasing a single photon. The rate of this reaction depends on the calcium concentration to which the photoprotein is exposed. Intact aequorin with coelenterazine has been used to monitor calcium flux in cell-based assays. Obelin is a 22-kDa monomeric protein that also requires coelenterazine for signal generation. Construction of an aequorin PCA or an obelin PCA would enable assays in which photon release only occurs if the reporter fragments are associated as a result of a ligand-protein interaction or a protein-protein interaction. Such an assay would combine measures of pathway activation with calcium flux, making the assays extraordinarily sensitive for pathway-based studies.

Although small monomeric reporters are preferred for this invention due to the small size of the reporter fragments, it will be apparent from the prior art that multimeric enzymes such as beta-galactosidase, beta-glucuronidase, tyrosinase, and other reporters can also be used in the present invention. A number of multimeric enzymes suitable for PCA have previously been described (U.S. Pat. No. 6,270,964). Fragments of multimeric proteins can be engineered using the principles of PCA described in the prior art; alternatively, naturally-occurring fragments or low-affinity subunits of multimeric enzymes can be used including the widely-used beta-galactosidase cc and co complementation systems. Beta-galactosidase (beta-gal) is a multimeric enzyme which forms tetramers and octomeric complexes of up to 1 million Daltons. Beta-gal subunits undergo self-oligomerization which leads to activity. This naturally-occurring phenomenon has been used to develop a variety of in vitro, homogeneous assays that are the subject of over 30 patents. Alpha- or omega-complementation of beta-gal, which was first reported in 1965, has been utilized to develop assays for the detection of antibody-antigen, drug-protein, protein-protein, and other bio-molecular interactions. The background activity due to self-oligomerization has been overcome in part by the development of low-affinity, mutant subunits with a diminished or negligible ability to complement naturally, enabling various assays including for example the detection of ligand-dependent activation of the EGF receptor in live cells (Rossi and Blau). These low-affinity subunits can be used to construct assays in conjunction with the present invention.

For some pathways, activation of the pathway leads to the translocation of a pre-existing protein-protein complex from one sub-cellular compartment to another, without an increase in the total number of protein-protein complexes. In that case, the fluorescent signal generated by the reassembled reporter at the site of complex formation within the cell can be imaged, allowing the trafficking of the complex to be monitored. Such “high-content” PCAs can be engineered for any suitable reporter for which the signal remains at the site of the protein-protein complex. Examples include the DHFR PCA, which has been used for high-content assays of signal transduction pathways (I Remy & S Michnick, 2001, Visualization of Biochemical Networks in Living Cells, Proc Natl Acad Sci USA, 98: 7678-7683) and also for high-throughput assays (I Remy et al., 1999, Erythropoietin receptor activation by a ligand-induced conformation change, Science 283: 990-993). Reconstituted DHFR binds methotrexate (MTX); if the MTX is conjugated to a fluorophore such as fluorescein, Texas Red, or BODIPY, the PCA signal can be localized within cells. Additional reporters particularly useful for high-content assays are described in U.S. Pat. No. 6,270,964 and include the green fluorescent protein (GFP) from Aequorea victoria. PCAs based on GFP, YFP, and other inherently fluorescent, luminescent or phosphorescent protein reporters are preferred embodiments of the present invention. Any number of fluorescent proteins have been described in the scientific literature (e.g. R Y Tsien, 1998, The Green Fluorescent Protein, in: Annual Reviews of Biochemistry 67: 509-544; J Zhang et al., 2000, Creating new fluorescent probes for cell biology, Nature Reviews 3: 906-918). Any mutant fluorescent protein can be engineered into fragments for use in the present invention. Suitable reporters include YFP, CFP, dsRed, mRFP, ‘citrine’, BFP, PA-GFP, ‘Venus’, SEYFP and other AFPs; and the red and orange-red fluorescent proteins from Anemonia and Anthozoa.

Reporters generating a high signal-to-background are preferred for the present invention. For example, PCAs based on YFP, SEYFP, or ‘Venus’ (T Nagai et al., 2002, A variant of yellow fluorescent protein with fast and efficient maturation for cell-biological applications, Nature Biotech. 20: 87-90) are particularly suitable for the present invention. PCAs based on proteins for which the signal can be triggered, such as a kindling fluorescent protein (KFP1) (DM Chudakov et al., 2003, Kindling fluorescent proteins for precise in vivo photolabeling, Nat. Biotechnol. 21, 191-194), a photo-converting fluorescent protein such as Kaede (R Ando et al., 2002, An optical marker based on the uv-induced green-red photoconversion of a fluorescent protein, Proc. Natl. Acad. Sci. USA, 2002, 99 (20): 12651-12656), or a photoactivatable protein such as PA-GFP (GH Patterson et al., 2002, A photoactivatable GFP for selective labeling of proteins and cells, Science 297: 1873-1877) may have advantages, particularly in cases where it is necessary to capture very rapid signaling events. KFP1 is derived from a unique GFP-like chromoprotein asCP from the sea anemone Anemonia sulcata. asCP is initially nonfluorescent, but in response to intense green light irradiation it becomes brightly fluorescent (kindles) with emission at 595 nm. Kindled asCP relaxes back to the initial nonfluorescent state with a half-life of <10 seconds. Alternatively, fluorescence can be “quenched” instantly and completely by a brief irradiation with blue light. The mutant (asCP A148G, or KFP1) is capable of unique irreversible photoconversion from the nonfluorescent to a stable bright-red fluorescent form that has 30 times greater fluorescent intensity than the unkindled protein, making it particularly suitable for live cell PCAs.

Alternative techniques for measuring protein-protein interactions are equally suitable for this invention. The most widespread fluorescent, cell-based protein-protein interaction assays to date are based on the phenomenon of fluorescence resonance energy transfer (FRET) or bioluminescence resonance energy transfer (BRET). In a FRET assay the genes for two different fluorescent reporters, capable of undergoing FRET are separately fused to genes encoding of interest, and the fusion proteins are co-expressed in live cells. When a protein complex forms between the proteins of interest, the fluorophores are brought into proximity if the two proteins possess overlapping emission and excitation, emission of photons by a first, “donor” fluorophore, results in the efficient absorption of the emitted photons by the second, “acceptor” fluorophore. The FRET pair fluoresces with a unique combination of excitation and emission wavelengths that can be distinguished from those of either fluorophore alone in living cells. As specific examples, a variety of GFP mutants have been used in FRET assays, including cyan, citrine, enhanced green and enhanced blue fluorescent proteins. With BRET, a luminescent protein—for example the enzyme Renilla luciferase (RLuc)—is used as a donor and a green fluorescent protein (GFP) is used as an acceptor molecule. Upon addition of a compound that serves as the substrate for Rluc, the FRET signal is measured by comparing the amount of blue light emitted by Rluc to the amount of green light emitted by GFP. The ratio of green to blue increases as the two proteins are brought into proximity. Newer methods are in development to enable deconvolution of FRET from bleedthrough and from autofluorescence. In addition, fluorescence lifetime imaging microscopy (FLIM) eliminates many of the artifacts associates with quantifying simple FRET intensity. Alternative methods for the measurement of protein-protein interactions can also be used for this invention. These include assays based on split ubiquitin, as well as two-hybrid and three-hybrid assays and similar approaches for the detection and measurement of protein-protein complexes. Many of these systems have been adapted for the generation of fluorescence signals in intact cells.

Methods for the Detection of Post-Translational Modifications of Proteins

The present invention teaches that cell-based fluorescence or luminescence assays for post-translationally modified proteins can be used to identify new activities of drugs and new therapeutic uses for known drugs. By applying antibodies to fixed cells, one can measure the absolute level and the subcellular location of a particular protein or class of proteins, as well as specific post-translational modifications (e.g. phosphorylation, acetylation, ubiquitination, sumoylation, methylation, nitrosylation, glycosylation, myristoylation, palmitoylation, farnesylation, etc.) that occur in response to drug treatment. In making the present invention, cell-based assays using modification state-specific antibodies were used to monitor the dynamic changes that occur in cells in the presence of a drug of interest.

In addition to phospho-specific antibodies, other modification-state-specific antibodies can, in principle, be generated for any macromolecule that undergoes a post-translational modification in the cell. Such novel reagents can be used in conjunction with this invention. Such post-translational modifications include methylation, acetylation, farnesylation, glycosylation, myristylation, ubiquitination, sumoylation, and other post-translational modifications that may occur in response to drug effects.

Such post-translational modifications may be detected using antibodies in conjunction with immunofluorescence, as described herein; however, the method is not limited to the use of antibodies. It is important to note that the invention is not limited to specific reagents or classes or reagents, or protocols for their use. Alternative (non-antibody) probes of target or pathway activity can be used, so long as they (a) bind differentially upon a change in a macromolecule in a cell, such that they reflect a change in pathway activity, cell signaling, or cell state related to the effect of a drug; (b) can be washed out of the cell in the unbound state, so that bound probe can be detected over the unbound probe background; and (c) can be detected either directly or indirectly, e.g. with a fluorescent or luminescent method. A variety of organic molecules, peptides, ligands, natural products, nucleosides and other probes can be detected directly, for example by labeling with a fluorescent or luminescent dye or a quantum dot; or can be detected indirectly, for example, by immunofluorescence with the aid of an antibody that recognizes the probe when it is bound to its target. Such probes could include ligands, native or non-native substrates, competitive binding molecules, peptides, nucleosides, and a variety of other probes that bind differentially to their targets based on post-translational modification states of the targets. It will be appreciated by one skilled in the art that some methods and reporters will be better suited to different situations. Particular reagents, fixing and staining methods may be more or less optimal for different cell types and for different pathways or targets.

In addition to proteins, a variety of macromolecules are modified post-translationally, including DNA and lipids. Methylation of DNA is important in the sequence-specific and gene-specific regulation of transcription. Phosphorylation of lipids is important in the control of cell signaling; for example, the balance between inositol polyphosphates is crucial in regulating the level of the second messenger, inositol trisphosphate (IP3); and the fatty acid composition of phospholipids such as phosphatidylcholine, phosphatidylinositol and phosphatidylserine regulates membrane fluidity and permeability. As the toolbox of modification-state-specific reagent expands, such assays will be added into the panels we are constructing for pharmacological profiling.

TABLE 4
Examples of modification-state-specific reagents that may
be used in conjunction with the present invention

Akt (pS472/pS473), Phospho-Specific (PKBa) Antibodies
Caveolin (pY14), Phospho-Specific Antibodies
Cdk1/Cdc2 (pY15), Phospho-Specific Antibodies
eNOS (pS1177), Phospho-Specific Antibodies
eNOS (pT495), Phospho-Specific Antibodies
ERK1/2 (pT202/pY204), Phospho-Specific Antibodies (p44/42 MAPK)
FAK (pY397), Phospho-Specific Antibodies
IkBa (pS32/pS36), Phospho-Specific Antibodies
Integrin b3 (pY759), Phospho-Specific Antibodies
JNK (pT183/pY185), Phospho-Specific Antibodies
Lck (pY505), Phospho-Specific Antibodies
p38 MAPK (pT180/pY182), Phospho-Specific Antibodies
p120 Catenin (pY228), Phospho-Specific Antibodies
p120 Catenin (pY280), Phospho-Specific Antibodies
p120 Catenin (pY96), Phospho-Specific Antibodies
Paxillin (pY118), Phospho-Specific Antibodies
Phospholipase Cg (pY783), Phospho-Specific Antibodies
PKARIIb (pS114), Phospho-Specific Antibodies
14-3-3 Binding Motif Phospho-specific Antibodies
4E-BP1 Phospho-specific Antibodies
AcCoA Carboxylase (Acetyl CoA) Phospho-specific Antibodies
Adducin Phospho-specific Antibodies
AFX Phospho-specific Antibodies
AlK (Aurora 2) Phospho-specific Antibodies
Akt (PKB) Phospho-specific Antibodies
Akt (PKB) Substrate Phospho-specific Antibodies
ALK Phospho-specific Antibodies
AMPK alpha Phospho-specific Antibodies
AMPK beta1 Phospho-specific Antibodies
APP Phospho-specific Antibodies
Arg-X-Tyr/Phe-X-pSer Motif Phospho-specific Antibodies
Arrestin 1, beta Phospho-specific Antibodies
ASK1 Phospho-specific Antibodies
ATF-2 Phospho-specific Antibodies
ATM/ATR Substrate Phospho-specific Antibodies
Aurora 2 (AlK) Phospho-specific Antibodies
Bad Phospho-specific Antibodies
Bcl-2 Phospho-specific Antibodies
Bcr Phospho-specific Antibodies
Bim EL Phospho-specific Antibodies
BLNK Phospho-specific Antibodies
BMK1 (ERK5) Phospho-specific Antibodies
BRCA1 Phospho-specific Antibodies
Btk Phospho-specific Antibodies
C/EBP alpha Phospho-specific Antibodies
C/EBP beta Phospho-specific Antibodies
c-Abl Phospho-specific Antibodies
CAKb Phospho-specific Antibodies
Caldesmon Phospho-specific Antibodies
CaM Kinase II Phospho-specific Antibodies
Cas, p130 Phospho-specific Antibodies
Catenin, beta Phospho-specific Antibodies
Catenin, p120 Phospho-specific Antibodies
Caveolin 1 Phospho-specific Antibodies
Caveolin 2 Phospho-specific Antibodies
Caveolin Phospho-specific Antibodies
c-Cbl Phospho-specific Antibodies
CD117 (c-Kit) Phospho-specific Antibodies
CD19 Phospho-specific Antibodies
cdc2 p34 Phospho-specific Antibodies
cdc2 Phospho-specific Antibodies
cdc25 C Phospho-specific Antibodies
cdk1 Phospho-specific Antibodies
cdk2 Phospho-specific Antibodies
CDKs Substrate Phospho-specific Antibodies
CENP-A Phospho-specific Antibodies
c-erbB-2 Phospho-specific Antibodies
Chk1 Phospho-specific Antibodies
Chk2 Phospho-specific Antibodies
c-Jun Phospho-specific Antibodies
c-Kit (CD117) Phospho-specific Antibodies
c-Met Phospho-specific Antibodies
c-Myc Phospho-specific Antibodies
Cofilin 2 Phospho-specific Antibodies
Cofilin Phospho-specific Antibodies
Connexin 43 Phospho-specific Antibodies
Cortactin Phospho-specific Antibodies
CPI-17 Phospho-specific Antibodies
cPLA2 Phospho-specific Antibodies
c-Raf (Raf1) Phospho-specific Antibodies
CREB Phospho-specific Antibodies
c-Ret Phospho-specific Antibodies
CrkII Phospho-specific Antibodies
CrkL Phospho-specific Antibodies
Cyclin B1 Phospho-specific Antibodies
DARPP-32 Phospho-specific Antibodies
DNA-topoisomerase II alpha Phospho-specific Antibodies
Dok-2, p56 Phospho-specific Antibodies
eEF2 Phospho-specific Antibodies
eEF2k Phospho-specific Antibodies
EGF Receptor (EGFR) Phospho-specific Antibodies
elF2 alpha Phospho-specific Antibodies
elF2B epsilon Phospho-specific Antibodies
elF4 epsilon Phospho-specific Antibodies
elF4 gamma Phospho-specific Antibodies
Elk-1 Phospho-specific Antibodies
eNOS Phospho-specific Antibodies
EphA3 Phospho-specific Antibodies
Ephrin B Phospho-specific Antibodies
erbB-2 Phospho-specific Antibodies
ERK1/ERK2 Phospho-specific Antibodies
ERK5 (BMK1) Phospho-specific Antibodies
Estrogen Receptor alpha (ER-a) Phospho-specific Antibodies
Etk Phospho-specific Antibodies
Ezrin Phospho-specific Antibodies
FADD Phospho-specific Antibodies
FAK Phospho-specific Antibodies
FAK2 Phospho-specific Antibodies
Fc gamma RIIb Phospho-specific Antibodies
FGF Receptor (FGFR) Phospho-specific Antibodies
FKHR Phospho-specific Antibodies
FKHRL1 Phospho-specific Antibodies
FLT3 Phospho-specific Antibodies
FRS2-alpha Phospho-specific Antibodies
Gab1 Phospho-specific Antibodies
Gab2 Phospho-specific Antibodies
GABA B Receptor Phospho-specific Antibodies
GAP-43 Phospho-specific Antibodies
GATA4 Phospho-specific Antibodies
GFAP Phospho-specific Antibodies
Glucocorticoid Receptor Phospho-specific Antibodies
GluR1 (Glutamate Receptor 1) Phospho-specific Antibodies
GluR2 (Glutamate Receptor 2) Phospho-specific Antibodies
Glycogen Synthase Phospho-specific Antibodies
GRB10 Phospho-specific Antibodies
GRK2 Phospho-specific Antibodies
GSK-3 alpha/beta Phospho-specific Antibodies
GSK-3 alpha Phospho-specific Antibodies
GSK-3 beta (Glycogen Synthase Kinase) Phospho-specific Antibodies
GSK-3 beta Phospho-specific Antibodies
GSK-3 Phospho-specific Antibodies
H2A.X Phospho-specific Antibodies
Hck Phospho-specific Antibodies
HER-2 (ErbB2) Phospho-specific Antibodies
Histone H1 Phospho-specific Antibodies
Histone H2A.X Phospho-specific Antibodies
Histone H2B Phospho-specific Antibodies
Histone H3 Phospho-specific Antibodies
HMGN1 (HMG-14) Phospho-specific Antibodies
Hsp27 (Heat Shock Protein 27) Phospho-specific Antibodies
IkBa (I kappa B-alpha) Phospho-specific Antibodies
Integrin alpha-4 Phospho-specific Antibodies
Integrin beta-1 Phospho-specific Antibodies
Integrin beta-3 Phospho-specific Antibodies
IR (Insulin Receptor) Phospho-specific Antibodies
IR/IGF1R (Insulin/Insulin-Like Growth Factor-1 Receptor)
Phospho-specific Antibodies
IRS-1 Phospho-specific Antibodies
IRS-2 Phospho-specific Antibodies
Jak1 Phospho-specific Antibodies
Jak2 Phospho-specific Antibodies
JNK (SAPK) Phospho-specific Antibodies
Jun Phospho-specific Antibodies
KDR Phospho-specific Antibodies
Keratin 18 Phospho-specific Antibodies
Keratin 8 Phospho-specific Antibodies
Kinase Substrate Phospho-specific Antibodies
Kip1, p27 Phospho-specific Antibodies
LAT Phospho-specific Antibodies
Lck Phospho-specific Antibodies
Leptin Receptor Phospho-specific Antibodies
LKB1 Phospho-specific Antibodies
Lyn Phospho-specific Antibodies
MAP Kinase/CDK Substrate Phospho-specific Antibodies
MAP Kinase, p38 Phospho-specific Antibodies
MAP Kinase, p44/42 Phospho-specific Antibodies
MAPKAP Kinase 1a (Rsk1) Phospho-specific Antibodies
MAPKAP Kinase 2 Phospho-specific Antibodies
MARCKS Phospho-specific Antibodies
Maturation Promoting Factor (MPF) Phospho-specific Antibodies
M-CSF Receptor Phospho-specific Antibodies
MDM2 Phospho-specific Antibodies
MEK1/MEK2 Phospho-specific Antibodies
MEK1 Phospho-specific Antibodies
MEK2 Phospho-specific Antibodies
MEK4 Phospho-specific Antibodies
MEK7 Phospho-specific Antibodies
Met Phospho-specific Antibodies
MKK3/MKK6 Phospho-specific Antibodies
MKK4 (SEK1) Phospho-specific Antibodies
MKK7 Phospho-specific Antibodies
MLC Phospho-specific Antibodies
MLK3 Phospho-specific Antibodies
Mnk1 Phospho-specific Antibodies
MPM2 Phospho-specific Antibodies
MSK1 Phospho-specific Antibodies
mTOR Phospho-specific Antibodies
Myelin Basic Protein (MBP) Phospho-specific Antibodies
Myosin Light Chain 2 Phospho-specific Antibodies
MYPT1 Phospho-specific Antibodies
neu (Her2) Phospho-specific Antibodies
Neurofilament Phospho-specific Antibodies
NFAT1 Phospho-specific Antibodies
NF-kappa B p65 Phospho-specific Antibodies
Nibrin (p95/NBS1) Phospho-specific Antibodies
Nitric Oxide Synthase, Endothelial (eNOS) Phospho-specific Antibodies
Nitric Oxide Synthase, Neuronal (nNOS) Phospho-specific Antibodies
NMDA Receptor 1 (NMDAR1) Phospho-specific Antibodies
NMDA Receptor 2B (NMDA NR2B) Phospho-specific Antibodies
nNOS Phospho-specific Antibodies
NPM Phospho-specific Antibodies
Opioid Receptor, delta Phospho-specific Antibodies
Opioid Receptor, mu Phospho-specific Antibodies
p53 Phospho-specific Antibodies
PAK1/2/3 Phospho-specific Antibodies
PAK2 Phospho-specific Antibodies
Paxilin Phospho-specific Antibodies
Paxillin Phospho-specific Antibodies
PDGF Receptor alpha/beta Phospho-specific Antibodies
PDGF Receptor alpha Phospho-specific Antibodies
PDGF Receptor beta Phospho-specific Antibodies
PDGFRb (Platelet Derived Growth Factor Receptor beta)
Phospho-specific Antibodies
PDK1 Docking Motif Phospho-specific Antibodies
PDK1 Phospho-specific Antibodies
PDK1 Substrate Phospho-specific Antibodies
PERK Phospho-specific Antibodies
PFK-2 Phospho-specific Antibodies
Phe Phospho-specific Antibodies
Phospholamban Phospho-specific Antibodies
Phospholipase C gamma-1 Phospho-specific Antibodies
Phosphotyrosine IgG Phospho-specific Antibodies
phox, p40 Phospho-specific Antibodies
PI3K Binding Motif, p85 Phospho-specific Antibodies
Pin1 Phospho-specific Antibodies
PKA Substrate Phospho-specific Antibodies
PKB (Akt) Phospho-specific Antibodies
PKB (Akt) Substrate Phospho-specific Antibodies
PKC alpha/beta II Phospho-specific Antibodies
PKC alpha Phospho-specific Antibodies
PKC delta/theta Phospho-specific Antibodies
PKC delta Phospho-specific Antibodies
PKC epsilon Phospho-specific Antibodies
PKC eta Phospho-specific Antibodies
PKC gamma Phospho-specific Antibodies
PKC Phospho-specific Antibodies
PKC Substrate Phospho-specific Antibodies
PKC theta Phospho-specific Antibodies
PKC zeta/lambda Phospho-specific Antibodies
PKD (PKC mu) Phospho-specific Antibodies
PKD2 Phospho-specific Antibodies
PKR Phospho-specific Antibodies
PLC beta 3 Phospho-specific Antibodies
PLC gamma 1 Phospho-specific Antibodies
PLC gamma 2 Phospho-specific Antibodies
PLD1 Phospho-specific Antibodies
PP1 alpha Phospho-specific Antibodies
PP2A Phospho-specific Antibodies
PPAR Alpha Phospho-specific Antibodies
PRAS40 Phospho-specific Antibodies
Presenilin-2 Phospho-specific Antibodies
PRK2 (pan-PDK1 phosphorylation site) Phospho-specific Antibodies
Progesterone Receptor Phospho-specific Antibodies
Protein Kinase A, RII (PKARII) Phospho-specific Antibodies
Protein Kinase B Phospho-specific Antibodies
Protein Kinase B Substrate Phospho-specific Antibodies
Protein Kinase C, alpha (PKCa) Phospho-specific Antibodies
Protein Kinase C, epsilon (PKCe) Phospho-specific Antibodies
PTEN Phospho-specific Antibodies
Pyk2 Phospho-specific Antibodies
Rac1/cdc42 Phospho-specific Antibodies
Rac-Pk Phospho-specific Antibodies
Rac-Pk Substrate Phospho-specific Antibodies
Rad 17 Phospho-specific Antibodies
Rad17 Phospho-specific Antibodies
Raf-1 Phospho-specific Antibodies
Ras-GRF1 Phospho-specific Antibodies
Rb (Retinoblastoma Protein) Phospho-specific Antibodies
Ret Phospho-specific Antibodies
Ribosomal Protein S6 Phospho-specific Antibodies
RNA polymerase II Phospho-specific Antibodies
Rsk, p90 Phospho-specific Antibodies
Rsk1 (MAPKAP K1a) Phospho-specific Antibodies
Rsk3 Phospho-specific Antibodies
S6 Kinase Phospho-specific Antibodies
S6 Kinase, p70 Phospho-specific Antibodies
S6 peptide Substrate Phospho-specific Antibodies
SAPK (JNK) Phospho-specific Antibodies
SAPK2 (Stress-activated Protein Kinase, SKK3, MKK3)
Phospho-specific Antibodies
SEK1 (MKK4) Phospho-specific Antibodies
Serotonin N-AT Phospho-specific Antibodies
Serotonin-N-AT Phospho-specific Antibodies
SGK Phospho-specific Antibodies
Shc Phospho-specific Antibodies
SHIP1 Phospho-specific Antibodies
SHP-2 Phospho-specific Antibodies
SLP-76 Phospho-specific Antibodies
Smad1 Phospho-specific Antibodies
Smad2 Phospho-specific Antibodies
SMC1 Phospho-specific Antibodies
SMC3 Phospho-specific Antibodies
SOX-9 Phospho-specific Antibodies
Src Family Negative Regulatory Site Phospho-specific Antibodies
Src Family Phospho-specific Antibodies
Src Phospho-specific Antibodies
Stat1 Phospho-specific Antibodies
Stat2 Phospho-specific Antibodies
Stat3 Phospho-specific Antibodies
Stat4 Phospho-specific Antibodies
Stat5 Phospho-specific Antibodies
Stat5A/Stat5B Phospho-specific Antibodies
Stat5ab Phospho-specific Antibodies
Stat6 Phospho-specific Antibodies
Syk Phospho-specific Antibodies
Synapsin Phospho-specific Antibodies
Synapsin site 1 Phospho-specific Antibodies
Tau Phospho-specific Antibodies
Tie 2 Phospho-specific Antibodies
Trk A Phospho-specific Antibodies
Troponin I, Cardiac Phospho-specific Antibodies
Tuberin Phospho-specific Antibodies
Tyk 2 Phospho-specific Antibodies
Tyrosine Hydroxylase Phospho-specific Antibodies
Tyrosine Phospho-specific Antibodies
VASP Phospho-specific Antibodies
Vav1 Phospho-specific Antibodies
Vav3 Phospho-specific Antibodies
VEGF Receptor 2 Phospho-specific Antibodies
Zap-70 Phospho-specific Antibodies

Instrumentation

The assays described above generate optically detectable signals that can be read on commercially available instrumentation, including fluorescence plate readers, luminometers, and flow cytometers. Such instrumentation is widely available from commercial manufacturers, including Molecular Devices, Packard, Perkin Elmer, Becton Dickinson, Beckman Coulter, and others. All such assays can be constructed in multiwell (96-well and 384-well) formats. The high-content assays described above, including the protein-fragment complementation assays 50 and immunofluorescence assays, generate optically detectable signals that can be spatially resolved within subcellular compartments. The resulting images can be captured with automated microscopes, confocal imaging systems, and similar devices. Suitable imaging instrumentation is widely available from a variety of commercial manufacturers including Molecular Devices (Universal Imaging), Amersham Bioscience, Cellomics, Evotec, Zeiss, Q3DM, Atto, and others. Image analysis software such as MetaMorph, the publicly available IMAGE software from the National Institutes of Health (http://rsb.info.nih.gov/nih-image/) and various proprietary software packages are used to distinguish the signal emanating from different subcellular compartments (membrane, cytosol, nucleus) and to quantitate the total fluorescence per cell. In addition, multi-well PCA formats for the present invention can be constructed for array-based or slide-based assay formats (Sabatini et al.) allowing the rapid, simultaneous processing of a large number of different PCAs on a single array.

Suitable pairs of interacting molecules for assay construction can be identified by any one of the methods outlined in FIG. 1. PCA enables a systematic characterization of the interactions made among the proteins in living cells by first examining whether different pairs of proteins generate a PCA signal in a cell type of interest and then determining whether the signal amount or subcellular location is affected by drugs that modify cell signaling. Systematic screening can also be performed to identify pathway elements; for example, a protein tagged with F1 of a suitable reporter can be tested individually against other proteins tagged with complementary fragment F2 (gene-by-gene analysis). The presence of a PCA signal indicates an interaction between the two proteins tagged with the complementary fragments. The advantage of the present invention is that, once an interaction has been identified, an assay is in hand that can be used to screen for drugs that modulate the pathway of interest by using a high-content or high-throughput PCA as a screen.

The components of many important cellular pathways and disease-related pathways have been partially elucidated, and the known or hypothesized interactions can readily be used to design assays according to the present invention. The present invention encompasses assays for a variety of steps in cancer-related pathways. A few of these steps are listed in Table 1. Any of the protein-protein interactions reported to date can be used as the basis for the construction of protein-fragment complementation assays enzyme-fragment complementation assays, FRET or BRET assays. All of the assays that are the subject of the present invention are of general use as validation assays or in basic experimental biology research as well as in drug discovery.

The following patents, published patent applications as well as all their foreign counterparts and all cited references therein are incorporated in their entirety by reference herein as if those references were denoted in the text:

US 20040161787 protein fragment complementation assays for high-throughput and high-content screening

US 20040137528 fragments of fluorescent proteins for protein fragment complementation assays

US 20040038298 protein fragment complementation assays for the detection of biological or drug interactions

US 20030108869 protein fragment complementation assay (PCA) for the detection of protein-protein, protein-small molecule and protein nucleic acid interactions based on the E. coli TEM-1 beta-lactamase

US 20030049688 protein fragment complementation assays for the detection of biological or drug interactions

US 20020064769 dynamic visualization of expressed gene networks in living cells

US 20010047526 mapping molecular interactions in plants with protein fragments complementation assays

U.S. Pat. No. 6,428,951 protein fragment complementation assays for the detection of biological or drug interactions

U.S. Pat. No. 6,294,330 protein fragment complementation assays for the detection of biological or drug interactions

U.S. Pat. No. 6,270,964 protein fragment complementation assays for the detection of biological or drug interactions

What is claimed is:

1. A method for identifying a new therapeutic use for a test entity, wherein said test entity is a drug or a drug candidate, said method comprising (A) selecting a test entity; (B) testing the activity of said test entity against a protein complex in a cell; (C) using the results obtained from (B) to identify a new activity of said test entity.

2. A method for screening a test entity, wherein said test entity is a drug or a drug candidate, said method comprising: (a) constructing an assay for a protein complex in a cell; (b) testing the effects of said test entity on one or more properties of said assay(s); (c) using the results of (b) to identify an activity of said test entity.

3. A method for identifying an entity that modulates the activity of a cellular pathway, said method comprising: (a) providing (i) a test entity and (ii) an assay for a protein complex in a cell; (b) contacting said assay with said test entity; and (c) detecting or measuring one or more properties of said assay; wherein a change in one or more properties of said assay in the presence of said test entity, relative to the absence of said test entity, indicates that the test entity modulates the activity of said cellular pathway.

4. A method according to any of claims 1-3 wherein at least one assay property that is measured is (a) the amount of said protein complex or (b) the subcellular location of said protein complex.

5. A method according to any of claims 1-3 wherein said test entity produces either (a) an overall increase in said protein complex, (b) an overall decrease in said protein complex, (c) a change in the subcellular location of said protein complex or (d) a change in the subcellular amount of said protein complex.

6. A method according to any of claims 1-3 wherein a property of said protein complex is altered by said test entity via one or more of the following mechanisms: (a) increasing or decreasing the formation of said complex; (b) stabilizing or destabilizing said complex; (c) increasing or decreasing the dissociation of said complex; (d) increasing or decreasing the rate of degradation or proteolysis of one or more proteins in said complex; (e) increasing or decreasing or modifying the post-translational modification status of one or more proteins in said complex; (f) increasing or decreasing the amount of one or more proteins in said complex.

7. A method for identifying a new therapeutic use for a test entity, wherein said test entity is a drug or drug candidate, said method comprising (A) selecting a test entity; (B) testing the ability of said test entity to alter the amount and/or post-translational modification status of one or more proteins in a cell or a collection of cells; (C) using the results obtained from (B) to identify a new activity of said test entity.

8. A method for screening a test entity, wherein said test entity is a drug, said method comprising: (a) constructing an assay for the amount and/or post-translational modification status of one or more proteins in a cell or a collection of cells; (b) testing the effects of said test entity on one or more properties of said assay(s); (c) using the results of (b) to identify an activity of said test entity.

9. A method for identifying an entity that modulates the activity of a cellular pathway, said method comprising: (a) providing (i) a test entity and (ii) an assay for the amount and/or post-translational modification status of one or more proteins in a cell or a collection of cells; (b) contacting said assay with said test entity; and (c) detecting or measuring one or more properties of said assay; wherein a change in one or more properties of said assay in the presence of said test entity, relative to the absence of said test entity, indicates that the test entity modulates the activity of said cellular pathway.

10. A method according to any of claims 1-3 or claims 7-9 wherein said test entity is a drug that is either (a) approved by a governmental regulatory agency for administration to a patient or (b) not approved by a governmental regulatory agency.

11. A method according to any of claims 1-3 or claims 7-9 wherein an immunofluorescence assay is used

12. A method according to any of claims 1-3 or claims 7-9 wherein at least one assay property that is detected or measured is selected from the group comprising (a) the phosphorylation status of one or more proteins; (b) the ubiquitination status of one or more proteins; (c) the sumoylation status of one or more proteins; (d) the methylation status of one or more proteins; (e) the acetylation status of one or more proteins; (f) the nitrosylation status of one or more proteins; (g) the myristoylation status of one or more proteins; (h) the palmitoylation status of one or more proteins; (i) the farnesylation status of one or more proteins; (j) the geranylation status of one or more proteins; or (k) the glycosylation status of one or more proteins.

13. A method according to any of claims 1-3 or claims 7-9 wherein said test entity results in an increase or decrease in the level or type of one or more post-translational modifications of one or more proteins, relative to the level or type of said post-translational modifications of said protein(s) in the absence of said test entity.

14. A method for either (a) reducing the proliferation of a mammalian cell or (b) increasing the proliferation of a mammalian cell, said method comprising administering to a patient an entity that modulates either the amount, the subcellular location, or the post-translational modification status of a protein complex in said cell, such that the entity modulates intracellular signaling in said cell.

15. A method according to any of claims 13-14 wherein said entity is a drug that is either approved by a governmental regulatory agency for administration to a patient or that is not approved by a governmental regulatory agency.

16. A method according to any of claims 13-14, said method comprising modulating a protein complex in a mammalian cell, said protein complex selected from the group consisting of (a) a RAS:RAF complex; (b) a PAK4:Cofilin complex; (c) a CDC42:PAK4 complex; (d) a CDC37:HSP90 complex; (e) a Cofilin1:LIMK2 complex; (f) a Cofilin1:PAK4 complex; (g) an EGFR:Grb2 complex; (h) a p53:E6 complex; (i) a p53:p53 complex; (j) a CDC25C:Chk1 complex; (k) a BAD:BID complex; (k) a CDC2:WEE1 complex; (l) a BCL-xL:BAD complex; (m) a BCL-xL:BIK complex; (n) a Cdc2:CDC25C complex; (o) a Chk1:CDC25A complex; (p) a CyclinD:CDK4 complex; (q) a CyclinE:CDK2 complex; (r) an HSP90:Eef2k complex; (s) a MAX:MYC complex; (t) a Cdc2:p21 complex; (u) a p27:ubiquitin complex; (v) a protein:ubiquitin complex; (w) a p53:Chk1 complex; or (x) a Smad3:HDAC complex.

17. An assay for a protein complex or protein complexes, said assay comprising either (a) whole proteins or (b) domains of proteins, wherein at least one of said proteins is selected from the group consisting of CDC2, CDK4, PAK4, COFILIN, WEE1, BAD, BID, BIK, BCL-xL, Chk1, CDC25C, CDC25A, E6, EGFR, GRB2, RAS, RAF, CDC37, LIMK2, HSP90, AKT1, p27, UBIQUITIN, p53, Smad3, HDAC, MAX, MYC, ERK, CyclinD or Cyclin E.

18. A collection of cells or a cell line, wherein said cells or cell line contains (a) whole proteins or (b) domains of proteins, wherein said proteins or said domains of proteins are separately linked to complementary fragments of a reporter, and wherein at least one of said proteins is selected from the group consisting of: CDC2, CDK4, PAK4, COFILIN, WEE1, BAD, BID, BCL-xL, CHK1, CDC25C, E6, EGFR, GRB2, RAS, RAF, CDC37, LIMK2, HSP90, AKT1, p27, UBIQUITIN, p53, Smad3, HDAC, MAX, MYC, ERK, CyclinD or Cyclin E.

19. An assay according to any of claims 2, 3, or 18 wherein said assay is selected from the group comprising (a) a protein-fragment complementation assay, (b) an enzyme-fragment complementation assay, (c) a subunit complementation assay, (d) a fluorescence resonance energy transfer assay (FRET), (e) a bioluminescence resonance energy transfer assay (BRET), (f) a split ubiquitin assay, (g) a split intein assay, (h) a two-hybrid assay (i) a three-hybrid assay or (j) an immunofluorescence assay.

20. A method according to any of claims 1-3 or 18 wherein said proteins are detected or measured with one or more of the following methods: (a) a protein-fragment complementation assay, (b) an enzyme-fragment complementation assay, (c) a subunit complementation assay, (d) a fluorescence resonance energy transfer assay (FRET), (e) a bioluminescence resonance energy transfer assay, (f) a split ubiquitin assay, (g) a split intein assay, (h) a two-hybrid assay; (i) a three-hybrid assay; or (j) an immunofluorescence assay.

21. An assay according to any of claims 2-3 or 18 wherein said assays comprise one or more reporters selected from the group consisting of a green fluorescent protein (GFP), a yellow fluorescent protein (YFP), a blue fluorescent protein (BFP), a cyan fluorescent protein (CFP), a red fluorescent protein (RFP), a luciferase, a beta-galactosidase, a dihydrofolate reductase, an RNAse, an intein, a ubiquitin, a beta-lactamase, or a mutant of any of the foregoing reporters.

22. An assay according to any of claims 1-3, 7-9 or 18 said assay comprising the use of one or more of the following methods: fluorescence microscopy, flow cytometry, fluorescence spectroscopy, luminometry, and/or automated image analysis.

23. A method for identifying drug leads and useful pharmacophores from a known test entity, wherein said entity is a drug or a drug candidate, said method comprising (A) selecting an entity; (B) testing the activity of said entity against a protein complex in a cell; (C) using the results obtained from (B) to identify a new drug lead based on said entity.

24. A method for identifying a drug lead compound that modulates the activity of a cellular pathway, said method comprising: (a) assembling a library of candidate drugs that modulate a cellular pathway; (b) screening the library of candidate drugs that modulate a cellular pathway by providing (i) a library of candidate drugs and (ii) an assay for a protein complex or a post-translationally-modified protein in a cell; (c) contacting said assay with said candidate drugs; and (d) detecting or measuring one or more properties of said assay; wherein a change in one or more properties of said assay in the presence of said drug candidates, relative to the absence of said drug candidates, identifies a drug lead that modulates a cellular pathway.
